(19) United States
(12) Patent Application Publication
(10) Pub. No.: US 2023/0046278 A1
(43) Pub. Date: Feb. 16, 2023

(54) METHOD FOR COLOURING KERATIN MATERIAL, COMPRISING THE USE OF AN ORGANIC C1-C6 ALKOXYSILANE AND TWO STRUCTURALLY DIFFERENT CELLULOSE TYPES

(71) Applicant: Henkel AG & Co. KGaA, Duesseldorf (DE)

(72) Inventors: Rene KROHN, Norderstedt (DE); Thomas HIPPE, Appen (DE); Jessica BRENDER, Hamburg (DE); Stefan HOEPFNER, Hamburg (DE)

(73) Assignee: Henkel AG & Co. KGaA, Duesseldorf (DE)

(21) Appl. No.: 17/782,096

(22) PCT Filed: Oct. 19, 2020
(86) PCT No.: PCT/EP2020/079326

§ 371 Date: Jun. 2, 2022

(30) Foreign Application Priority Data
Dec. 4, 2019 (DE) 10 2019 218 860.5

(51) Int. Cl. A61K 8/58; A61Q 5/10; A61K 8/73; A61K 8/81

(52) U.S. Cl. CPC A61K 8/585; A61Q 5/10; A61K 8/731; A61K 8/8147; A61K 2800/95; A61K 2800/43; A61K 2800/882; A61K 2800/884

(57) ABSTRACT

It is an object of the present disclosure to provide a method for treating keratinous material, in particular human hair, wherein there is applied to the keratinous material
a first composition (A) comprising
(A1) one or more organic C₁-C₆ alkoxy silanes and/or condensation products thereof, and
   a second composition (B) comprising
(B1) a first cellulose and
(B2) a second cellulose different from the first cellulose (B1).

### CROSS-REFERENCE TO RELATED APPLICATION

This application is a U.S. National-Stage entry under 35 U.S.C. § 371 based on International Application No. PCT/EP2020/079326, filed Oct. 19, 2020, which was published under PCT Article 21(2) and which claims priority to German Application No. 102019218860.5, filed Dec. 4, 2019, which are all hereby incorporated in their entirety by reference.

### TECHNICAL FIELD

The present application is in the field of cosmetics and concerns a method for the treatment of keratinous material, in particular human hair, which comprises the use of two compositions (A) and (B). Composition (A) is a preparation comprising at least one C₁-C₆ organic alkoxysilane, and composition (B) includes at least two structurally different celluloses (B1) and (B2).

A second object of the present disclosure is a multi-component packaging unit (kit-of-parts) for dyeing keratinous material, which comprises, separately packaged in two packaging units, the two compositions (A) and (B) described above

### BACKGROUND

Changing the shape and color of keratinous fibers, especially hair, is an important area of modern cosmetics. To change the color of the hair, the specialist knows various coloring systems, depending on the requirements of coloring. For permanent, intensive dyeings with good fastness properties and good gray coverage, oxidation dyes are usually used. Such colorants usually contain oxidation dye precursors, so-called developer components and coupler components, which form the actual dyes under the influence of oxidizing agents such as hydrogen peroxide. Oxidation dyes are exemplified by very long-lasting dyeing results.

When using direct dyes, already formed dyes diffuse from the colorant into the hair fiber. Compared to oxidative hair dyeing, the dyeings obtained with direct dyes have lower durability and faster washout. Dyes with direct dyes usually remain on the hair for a period of between 5 and 20 washes.

For short-term color changes on the hair and/or skin, the use of color pigments is known. Color pigments are generally understood to be insoluble, color-imparting substances. These are present undissolved in the form of small particles in the coloring formulation and are merely deposited externally on the hair fibers and/or skin surface. Therefore, they can usually be removed without residue by a few washes with surfactant-comprising cleaning agents. Various products of this type are available on the market under the name of hair mascara.

If the user desires particularly long-lasting colorations, the use of oxidative colorants has so far been his/her only option. However, despite multiple optimization attempts, an unpleasant ammonia odor or amine odor cannot be completely avoided during oxidative hair coloring. The hair damage still associated with the use of the oxidative dyes also has a detrimental effect on the user's hair.

EP 2168633 B1 deals with the task of producing long-lasting hair colorations using pigments. The publication teaches that when a combination of pigment, organic silicon compound, hydrophobic polymer and a solvent is used on hair, it is possible to create colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

### BRIEF SUMMARY

This disclosure provides a method for treating keratinous material in which the following are applied to the keratinous material: a first composition (A) comprising (A1) one or more organic C1-C6 alkoxy silanes and/or condensation products thereof, and a second composition (B) comprising (B1) a first cellulose and (B2) a second cellulose different from the first cellulose (B1).

This disclosure also provides a multicomponent packaging unit (kit-of-parts) for treating keratinous material, comprising a separately prepared first container comprising a first composition (A), and second container comprising a second composition (B), wherein the first composition (A) comprises (A1) one or more organic C1-C6 alkoxy silanes and/or condensation products thereof, and the second composition (B) comprises (B1) a first cellulose and (B2) a second cellulose different from the first cellulose (B1), and optionally further comprising a separately assembled third container comprising a third composition (C), wherein the third composition (C) comprises at least one colorant compound chosen from pigments and/or direct dyes.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It is to be appreciated that all numerical values as provided herein are approximate values with endpoints or particular values intended to be read as “about” or “approximately” the value as recited.

When alkoxy silanes or their hydrolysis or condensation products are applied to keratinous material, a film or coating is formed on the keratinous material, which completely envelops the keratinous material and in this way strongly influences the properties of the keratinous material. Possible areas of application include permanent styling or permanent shape modification of keratin fibers. In this method, the keratin fibers are mechanically shaped into the desired form and then fixed in this form by forming the coating described above. Another particularly suitable application is the coloring of keratin material. In this application, the coating or film is produced in the presence of a colorant compound, for example a pigment. The film colored by the pigment remains on the keratin material or keratin fibers and results in surprisingly wash-resistant colorations.

The major advantage of the alkoxy silane-based dyeing principle is that the high reactivity of this class of compounds enables very fast coating. This means that good coloring results can be achieved even after short application periods of just a few minutes. In addition, the coating is formed on the surface of the keratin material and does not change the structure inside this keratin, so this dyeing technology is a very gentle method of changing the coloration of the keratin material.

However, dyeing methods that rely on the formation of dyed films or coatings are still in need of optimization. In particular, the color intensities and fastness properties of the dyeings obtained with this dyeing system can always be further improved. The manipulation, consistency and applicability of the formulations also still require optimization.

It was therefore the task of the present application to find a method for dyeing keratinous materials, in particular human hair, which has improved color intensities and improved fastness properties, in particular improved fastness to washing and improved fastness to rubbing. Furthermore, the formulations applied in this method should have improved manipulation, consistency and applicability.

Surprisingly, it has been found that this task can be fully solved if the keratin material is treated in a method in which two compositions (A) and (B) are applied to the keratin material. Here, the first composition (A) comprises at least one organic C₁-C₆ alkoxy silane (A1) and/or its condensation product, and the second composition (B) is exemplified by its content of at least two structurally different celluloses (B1) and (B2).

A first object of the present disclosure is a method for treating keratinous material, in particular human hair, wherein there is applied to the keratinous material
a first composition (A) comprising

(A1) one or more organic C₁-C₆ alkoxy silanes and/or condensation products thereof, and
a second composition (B) comprising

(B1) a first cellulose and

(B2) a second cellulose different from the first cellulose (B1).

In other words, a first object of the present disclosure is a method for treating keratinous material, in particular human hair, wherein there is applied to the keratinous material
a first composition (A) comprising

(A1) one or more organic C₁-C₆ alkoxy silanes and/or condensation products thereof, and
a second composition (B) comprising

(B1) a first cellulose and

(B2) a second cellulose that is structurally different from the first cellulose (B1).

When composition (A) was applied to the keratin material as part of a dyeing method, an improvement in color intensity, color fastness and rub fastness was observed, in particular, when compositions (A) and (B) were mixed with each other before application and added to the keratin material in their mixture. Even when composition (B) was applied to the keratin material in the form of an after-treatment agent after application of composition (A), very good results were obtained.

### Keratinous Material Coloring

Keratinous material means hair, the skin, the nails (such as fingernails and/or toenails). Furthermore, wool, fur and feathers also fall under the definition of keratinous material.

Preferably, keratinous material means human hair, human skin and human nails, in particular fingernails and toenails. Very preferably, keratinous material means human hair.

Organic C₁-C₆ Alkoxy Silanes (A1) and/or their Condensation Products in the Composition (A)

The composition (A) comprises one or more organic C₁-C₆ alkoxy silanes (A1) and/or their condensation products.

The organic C₁-C₆ alkoxy silane(s) are organic, non-polymeric silicon compounds, preferably selected from the group of silanes having one, two or three silicon atoms

Organic silicon compounds, alternatively referred to as organosilicon compounds, are compounds that either have a direct silicon-carbon (Si—C) bond or in which the carbon is attached to the silicon atom via an oxygen, nitrogen or sulfur atom. The organic silicon compounds of the present disclosure are preferably compounds comprising one to three silicon atoms. Particularly preferably, the organic silicon compounds contain one or two silicon atoms.

According to IUPAC rules, the term silane stands for a group of substances of chemical compounds based on a silicon structure and hydrogen. In organic silanes, the hydrogen atoms are wholly or partially replaced by organic groups such as (substituted) alkyl groups and/or alkoxy groups.

A characteristic feature of the C₁-C₆ alkoxy silanes of the present disclosure is that at least one C₁-C₆ alkoxy group is directly bonded to a silicon atom. The C₁-C₆ alkoxy silanes as contemplated herein thus comprise at least one structural unit R′R″R′″Si—O—(C₁-C₆ alkyl) where the radicals R′, R″ and R′″ represent the three remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolyzable groups per molecule. If the hydrolysable C₁-C₆ alkoxy group is an ethoxy group, the organic silicon compound preferably comprises a structural unit R′R″R′″Si—O—CH₂—CH₃. The radicals R′, R″, and R′″ again represent the three remaining free valences of the silicon atom.

Even the addition of small amounts of water leads first to hydrolysis and then to a condensation reaction between the organic alkoxy silanes. For this reason, both the organic alkoxy silanes (A1) and their condensation products may be present in the composition.

A condensation product is a product formed by reaction of at least two organic C₁-C₆ alkoxy silanes with elimination of water and/or with elimination of a C₁-C₆ alkanol.

The condensation products can be, for example, dimers, but also trimers or oligomers, with the condensation products being in equilibrium with the monomers.

Depending on the amount of water used or consumed in the hydrolysis, the equilibrium shifts from monomeric C₁-C₆ alkoxysilane to condensation product.

In a very particularly preferred embodiment, a method as contemplated herein is described in that the composition (A) comprises one or more organic C₁-C₆ alkoxy silanes (A1) selected from silanes having one, two or three silicon atoms, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A further particularly preferred method as contemplated herein is described wherein the composition (A) comprises one or more organic C₁-C₆ alkoxy silanes (A1) selected from the group of silanes having one, two or three silicon atoms, and wherein the C₁-C₆ alkoxy silanes further comprise one or more basic chemical functions.

Particularly good results were obtained when C₁-C₆ alkoxy silanes of the formula (S-I) and/or (S-II) were used in the method as contemplated herein. Since, as previously described, hydrolysis/condensation already starts at traces of moisture, the condensation products of the C₁-C₆ alkoxy silanes of formula (S-I) and/or (S-II) are also included in this embodiment.

In another very particularly preferred embodiment, a method as contemplated herein is described wherein the first composition (A) comprises one or more organic C₁-C₆ alkoxy silanes (A1) of the formula (S-I) and/or (S-II),

R₁R₂N-L-Si(OR₃)ₐ(R₄)_{b} (S-I)

where
R₁, R₂ independently represent a hydrogen atom or a C₁-C₆ alkyl group,
L is a linear or branched, divalent C₁-C₂₀ alkylene group,
R₃, R₄ are independent of each other for a C₁-C₆ alkyl group,
a represents an integer from 1 to 3, and
b is the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d}′(OR₅′)_{c}′ (S-II),

where
R₅, R₅′, R₅″, R₆, R₆′ and R₆″ independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of formula (S-III),

-(A″″)-Si(R₆″)_{d}″(OR₅″)_{c}″ (S-III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the radicals from e, f, g and h is different from 0, and/or their condensation products.

The substituents R₁, R₂, R₃, R₄, R₅, R₅′, R₅″, R₆, R₆′, R₆″, R₇, R₈, L, A, A′, A″, A′″ and A″″ in the compounds of formula (S-I) and (S-II) are exemplified below:

Examples of a C₁-C₆ alkyl group include methyl, ethyl, propyl, isopropyl, n-butyl, s-butyl and t-butyl, n-pentyl and n-hexyl groups. Propyl, ethyl and methyl are preferred alkyl radicals. Examples of a C₂-C₆ alkenyl group are vinyl, allyl, but-2-enyl, but-3-enyl as well as isobutenyl, preferred C₂-C₆ alkenyl radicals are vinyl and allyl. Preferred examples of a hydroxy-C₁-C₆-alkyl group include a hydroxymethyl, a 2-hydroxyethyl, a 2-hydroxypropyl, a 3-hydroxypropyl, a 4-hydroxybutyl, a 5-hydroxypentyl and a 6-hydroxyhexyl group; a 2-hydroxyethyl group is particularly preferred. Examples of an amino-C₁-C₆-alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH2-CH2-CH2-), and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched C₃-C₂₀ divalent alkylene groups are (—CH₂—CH(CH₃)—) and (—CH₂—CH(CH₃)—CH₂—).

In the organic silicon compounds of the formula (S-I)

R₁R₂N-L-Si(OR₃)ₐ(R₄)_{b} (S-I),

R₁ and R₂ independently represent a hydrogen atom or a C₁-C₆ alkyl group. Very preferably, R₁ and R₂ both represent a hydrogen atom.

In the middle part of the organic silicon compound is the structural unit or linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each -L- grouping may form two bonds.

Preferably, -L- represents a linear, divalent C₁-C₂₀ alkylene group. Further preferably, -L- represents a linear divalent C₁-C₆ alkylene group. Particularly preferably, -L- stands for a methylene group (—CH₂—), an ethylene group (—CH₂—CH₂—), a propylene group (—CH₂—CH₂—CH₂—) or a butylene group (—CH₂—CH₂—CH₂—CH₂—). Further preferably, L represents a propylene group (—CH₂—CH₂—CH₂—).

The organic silicon compounds of the formula (S-I) as contemplated herein

R₁R₂N-L-Si(OR₃)ₐ(R₄)_{b} (S-I),

carry the silicon-comprising grouping —Si(OR₃)ₐ(R₄)_{b} at one end.

In the terminal structural unit —Si(OR₃)ₐ(R₄)_{b}, R₃ and R₄ independently represent a C₁-C₆ alkyl group. Further preferably, R₃ and R₄ independently represent a methyl group or an ethyl group.

Here, a represents an integer from 1 to 3, and b represents the integer 3-a. If a represents the number 3, then b is 0. If a stands for the number 2, then b is equal to 1. If a stands for the number 1, then b is equal to 2.

Keratin treatment agents with particularly good properties could be prepared if the composition (A) comprises at least one organic C₁-C₆ alkoxy silane of the formula (S-I) in which the radicals R₃, R₄ independently of one another represent a methyl group or an ethyl group.

Furthermore, dyeings with the best color fastness could be obtained if the composition (A) comprises at least one organic C₁-C₆ alkoxy silane of the formula (S-I) in which the radical a represents the number 3. In this case, the radical b stands for the number 0.

In a further preferred embodiment, a method as contemplated herein is described wherein the composition (A) comprises one or more organic C₁-C₆ alkoxy silanes of the formula (S-I),

where
R₃, R₄ independently represent a methyl group or an ethyl group, and
a stands for the number 3 and
b stands for the number 0.

In a further preferred embodiment, a method as contemplated herein is described wherein the composition (A) comprises at least one or more organic C₁-C₆ alkoxy silanes of the formula (S-I),

R₁R₂N-L-Si(OR₃)ₐ(R₄)_{b} (S-I),

where
R₁, R₂ both represent a hydrogen atom, and
L is a linear, divalent C₁-C₆ alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

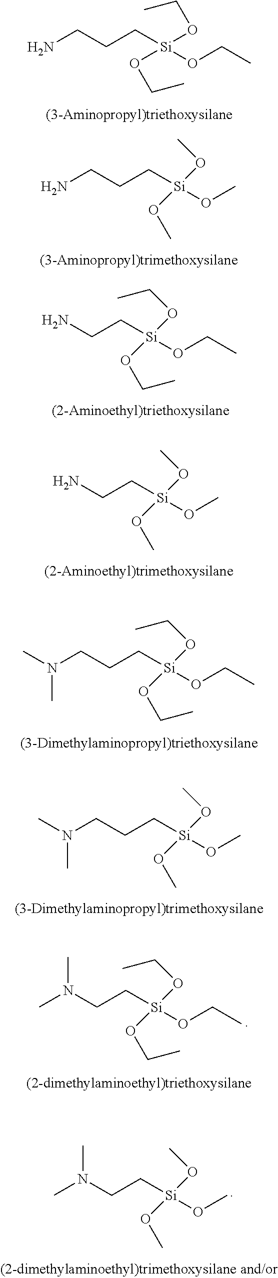

In a further preferred embodiment, a method as contemplated herein is described wherein the first composition (A) comprises at least one organic C₁-C₆ alkoxysilane (A1) of formula (S-I) selected from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane,
(2-Dimethylaminoethyl)trimethoxysilane
   and/or their condensation products.

The aforementioned organic silicon compounds of formula (I) are commercially available.

(3-Aminopropyl)trimethoxysilane is available for purchase from Sigma-Aldrich, for example.
(3-Aminopropyl)triethoxysilane is also commercially available from Sigma-Aldrich.

In a further embodiment of the method as contemplated herein, composition (A) may also comprise one or more organic C₁-C₆ alkoxy silanes of formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d}′(OR₅′)_{c}′ (S-II).

The organosilicon compounds of the formula (S-II) as contemplated herein each carry at their two ends the silicon-comprising groupings (R₅₀)_{c}(R₆)_{d}Si— and —Si(R₆′)_{d}′(OR₅′)_{c}′.

In the middle part of the molecule of formula (S-II) there are the groupings -(A)ₑ- and —[NR₇-(A′)]_{f}- and —[O-(A″)]_{g}- and —[NR₈-(A′″)]ₕ-. Here, each of the radicals e, f, g and h can independently represent the number 0 or 1, with the proviso that at least one of the radicals e, f, g and h is other than 0. In other words, an organic silicon compound of formula (II) as contemplated herein comprises at least one grouping selected from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d}′(OR₅′)_{c}′, the radicals R₅, R₅′, R₅″ independently represent a C₁-C₆ alkyl group. The radicals R₆, R₆′ and R₆″ independently represent a C₁-C₆ alkyl group.

Here c stands for an integer from 1 to 3, and d stands for the integer 3-c. If c stands for the number 3, then d is 0. If c stands for the number 2, then d is equal to 1. If c stands for the number 1, then d is equal to 2.

Similarly, c′ represents an integer from 1 to 3, and d′ represents the integer 3-c′. If c′ stands for the number 3, then d′ is equal to 0. If c′ stands for the number 2, then d′ is equal to 1. If c′ stands for the number 1, then d′ is equal to 2.

Dyes with the best color fastness could be obtained when the radicals c and c′ both stand for the number 3. In this case, d and d′ both stand for the number 0.

In a further preferred embodiment, a method as contemplated herein is described wherein the composition (A) comprises one or more organic C₁-C₆ alkoxy silanes of the formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d}′(OR₅′)_{c}′ (S-II),

where
R₅ and R₅′ independently represent a methyl group or an ethyl group,
c and c′ both stand for the number 3 and
d and d′ both stand for the number 0.

When c and c′ are both 3 and d and d′ are both 0, the organic silicon compounds as contemplated herein correspond to the formula (S-IIa)

(R₅O)₃Si-(A)ₑ-[NR₇-(A′)]_{f}-[O-(A″)]_{g}-[NR₈-(A′″)]ₕ-Si(OR₅′)₃ (S-IIa).

The radicals e, f, g, and h can independently represent the number 0 or 1, with at least one residue from e, f, g, and h being different from zero. The abbreviations e, f, g and h thus define which of the groupings -(A)ₑ- and —[NR₇-(A′)]_{f}- and —[O-(A″)]_{g}- and —[NR₈-(A′″)]ₕ- are located in the middle part of the organic silicon compound of formula (II).

In this context, the presence of certain groupings has proven to be particularly advantageous in terms of achieving washfast dyeing results. Particularly good results could be obtained if at least two of the radicals e, f, g and h stand for the number 1. Very preferably, e and f both stand for the number 1. Furthermore, g and h both represent the number 0.

When e and f are both 1 and g and h are both 0, the organic silicon compounds as contemplated herein are represented by the formula (S-IIb)

(R₅₀)_{c}(R₆)_{d}Si-(A)-[NR₇-(A′)]-Si(R₆′)_{d}′(OR₅′)_{c}′ (S-IIb).

Radicals A, A′, A″, A′″ and A″″ independently represent a linear or branched C₁-C₂₀ divalent alkylene group. Preferably, A, A′, A″, A′″ and A″″ independently represent a linear divalent C₁-C₂₀ alkylene group. Further preferably, A, A′, A″, A′″ and A″″ independently represent a linear divalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

Particularly preferably, the radicals A, A′, A″, A′″ and A″″ independently represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

When the radical f represents the number 1, the organic silicon compound of formula (II) as contemplated herein comprises a structural grouping —[NR₇-(A′)]-.

When the radical h represents the number 1, the organic silicon compound of formula (II) as contemplated herein comprises a structural grouping —[NR₈-(A′″)]-.

Here, R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III)

-(A″″)-Si(R₆″)ₐ″(OR₅″)_{c}″ (S-III).

Very preferably, R₇ and R₈ independently represent a hydrogen atom, a methyl group, a 2-hydroxyethyl group, a 2-alkenyl group, a 2-aminoethyl group or a group of formula (S-III).

When the radical f represents the number 1 and the radical h represents the number 0, the organic silicone compound as contemplated herein comprises the grouping [NR₇-(A′)] but not the grouping —[NR₈-(A′″)]. If the radical R₇ now stands for a grouping of the formula (III), the organic silicone compound comprises 3 reactive silane groups.

In a further preferred embodiment, a method as contemplated herein is described wherein the composition (A) comprises one or more organic C₁-C₆ alkoxy silanes (A1) of the formula (S-II)

(R₅O)_{c}(R₆)_{d}Si-(A)ₑ-[NR₇-(A′)]_{f}-[O-(A″)]_{g}-[NR₈-(A′″)]_{h-Si}(R₆′)_{d}′(OR₅′)_{c}′ (S-II),

where
e and f both stand for the number 1,
g and h both stand for the number 0,
A and A′ independently of one another represent a linear, divalent C₁-C₆ alkylene group and
R₇ represents a hydrogen atom, a methyl group, a 2-hydroxyethyl group, a 2-alkenyl group, a 2-aminoethyl group or a group of the formula (S-III).

In a further preferred embodiment, a method as contemplated herein is described wherein the composition (A) comprises one or more organic C₁-C₆ alkoxy silanes (A1) of formula (S-II), wherein
e and f both stand for the number 1,
g and h both stand for the number 0,
A and A′ independently represent a methylene group (—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of the formula (S-III).

Organic silicon compounds of the formula (S-II) which are well suited for solving the problem as contemplated herein are

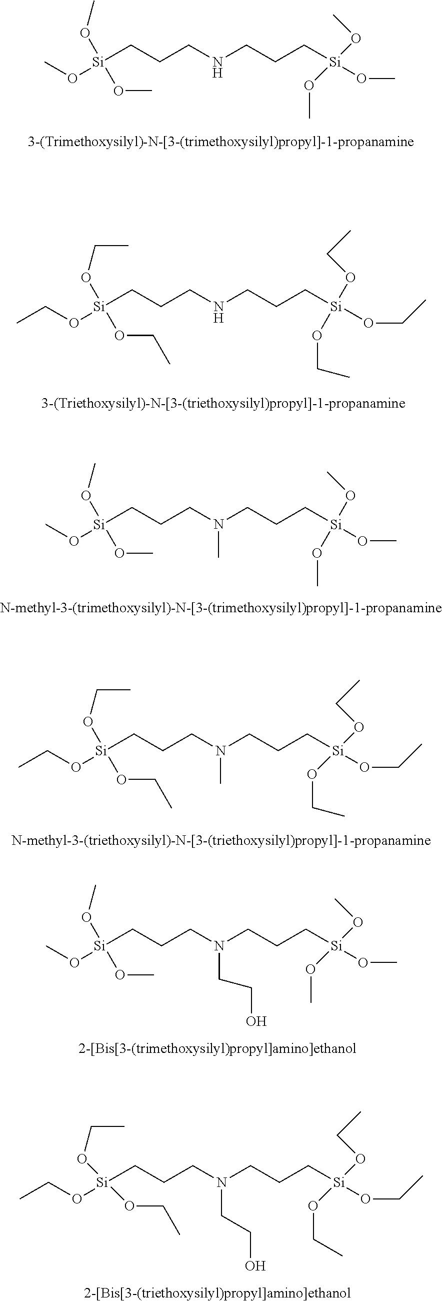

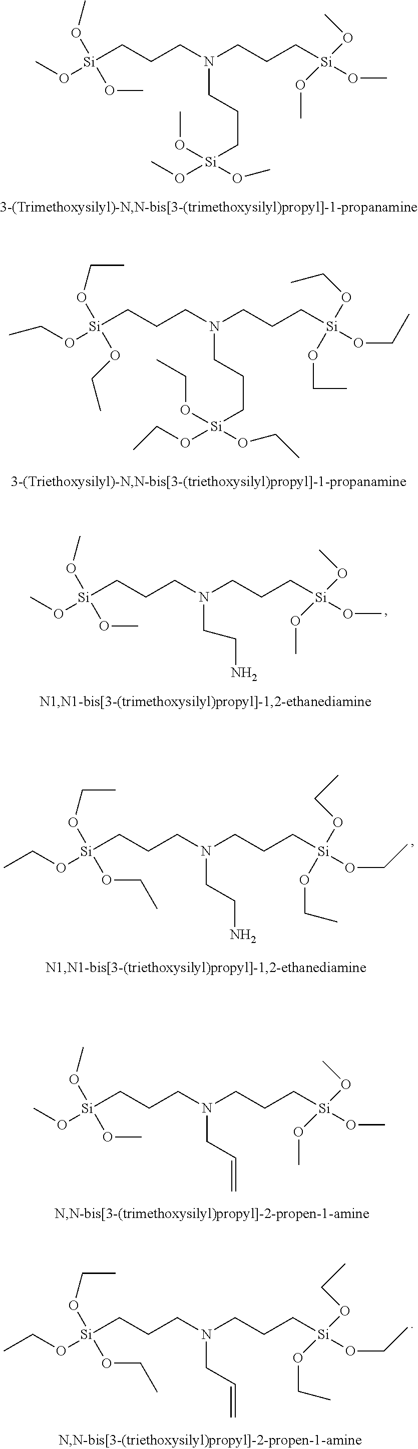

The aforementioned organic silicon compounds of formula (S-II) are commercially available.

Bis(trimethoxysilylpropyl)amines with CAS number 82985-35-1 can be purchased from Sigma-Aldrich, for example.

Bis[3-(triethoxysilyl)propyl]amines with CAS number 13497-18-2 can be purchased from Sigma-Aldrich, for example.

N-methyl-3-(trimethoxysilyl)-N-[3-(trimethoxysilyl)propyl]-1-propanamine is alternatively known as bis(3-trimethoxysilylpropyl)-N-methylamine and can be purchased commercially from Sigma-Aldrich or Fluorochem.

3-(Triethoxysilyl)-N,N-bis[3-(triethoxysilyl)propyl]-1-propanamine with CAS number 18784-74-2 can be purchased from Fluorochem or Sigma-Aldrich, for example.

In a further preferred embodiment, a method as contemplated herein is described wherein the composition (A) comprises one or more organic C₁-C₆ alkoxy silanes of formula (S-II) selected from the group of
3-(Trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]ethanol
2-[Bis[3-(triethoxysilyl)propyl]amino]ethanol
3-(Trimethoxysilyl)-N,N-bis[3-(trimethoxysilyl)propyl]-1-propanamine
3-(triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine,
   and/or their condensation products.

In further dyeing trials, it has also been found to be particularly advantageous that at least one organic C₁-C₆ alkoxy silane (A1) of the formula (S-IV) was used in the method as contemplated herein R₉Si(OR₁₀)ₖ(R₁₁)ₘ (S-IV).

The compounds of formula (S-IV) are organic silicon compounds selected from silanes having one, two or three silicon atoms, wherein the organic silicon compound comprises one or more hydrolyzable groups per molecule.

The organic silicon compound(s) of formula (S-IV) may also be referred to as silanes of the alkyl-C₁-C₆-alkoxy-silane type,

R₉Si(OR₁₀)ₖ(R₁₁)ₘ (S-IV),

where
R₉ represents a C₁-C₁₂ alkyl group,
R₁₀ stands for a C₁-C₆ alkyl group,
R₁₁ stands for a C₁-C₆ alkyl group
k is an integer from 1 to 3, and
m stands for the integer 3-k.

In a further embodiment, a particularly preferred method as contemplated herein is described

wherein the first composition (A) comprises one or more organic C₁-C₆ alkoxy silanes (A1) of the formula (S-IV),

R₉Si(OR₁₀)ₖ(R₁₁)ₘ (S-IV),

where
R₉ represents a C₁-C₁₂ alkyl group,
R₁₀ stands for a C₁-C₆ alkyl group,
R₁₁ stands for a C₁-C₆ alkyl group
k is an integer from 1 to 3, and
m stands for the integer 3-k,

and/or their condensation products.

In the organic C₁-C₆ alkoxy silanes of formula (S-IV), the radical R₉ represents a C1-C12 alkyl group. This C₁-C₁₂ alkyl group is saturated and can be linear or branched. Preferably, R₉ represents a linear C₁-C₈ alkyl group. Preferably, R₉ represents a methyl group, an ethyl group, an n-propyl group, an n-butyl group, an n-pentyl group, an n-hexyl group, an n-octyl group, or an n-dodecyl group. Further preferably, R₉ represents a methyl group, an ethyl group or an n-octyl group.

In the organic silicon compounds of formula (S-IV), the radical R₁₀ represents a C₁-C₆ alkyl group. Further preferably, R₁₀ represents a methyl group or an ethyl group.

In the organic silicon compounds of formula (S-IV), the radical R₁₁ represents a C₁-C₆ alkyl group. Further preferably, R₁₁ represents a methyl group or an ethyl group.

Furthermore, k stands for an integer from 1 to 3, and m stands for the integer 3-k. If k stands for the number 3, then m is 0. If k stands for the number 2, then m is equal to 1. If k stands for the number 1, then m is equal to 2.

Dyeings with the best color fastness could be obtained when the composition (A) comprises at least one organic C₁-C₆ alkoxy silane (A1) of formula (S-IV) in which the radical k represents the number 3. In this case, the radical m stands for the number 0.

Organic silicon compounds of the formula (S-IV) which are particularly suitable for solving the problem as contemplated herein are

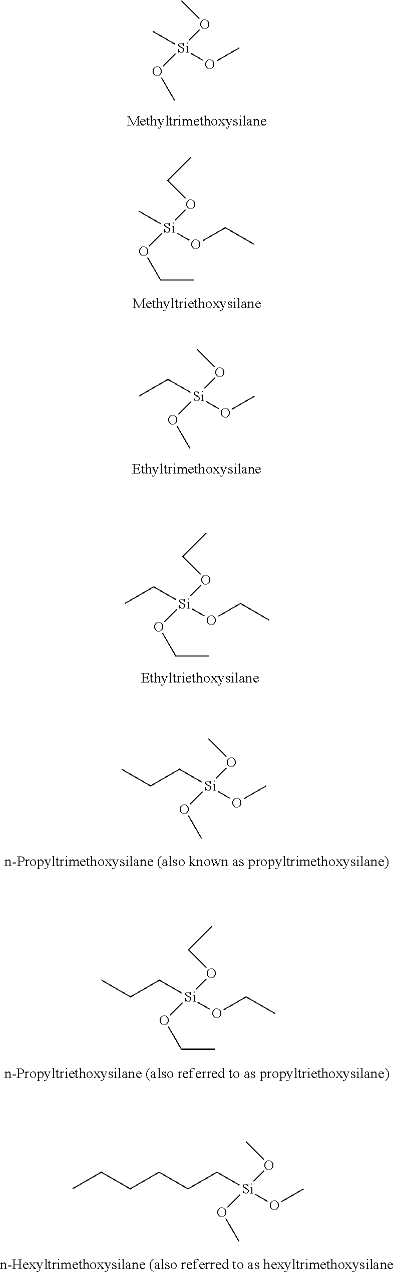

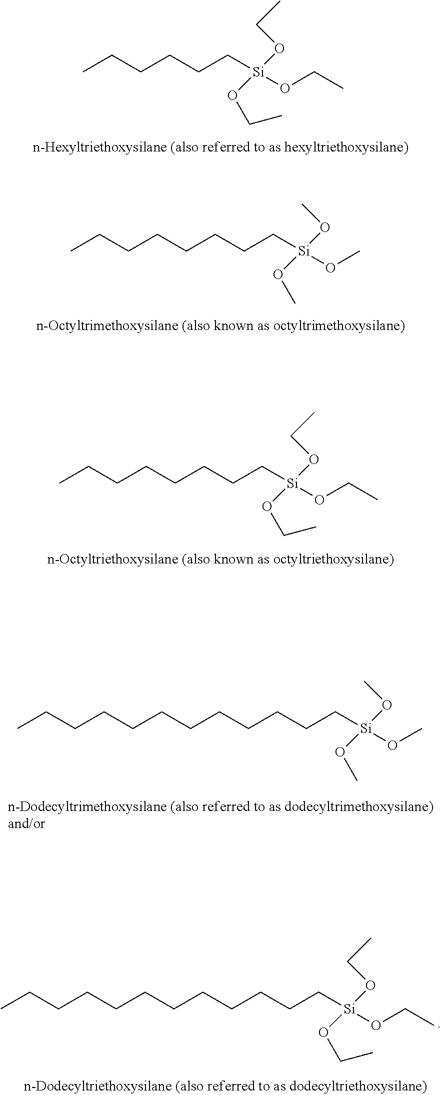

In a further preferred embodiment, a method as contemplated herein is described wherein the first composition (A) comprises at least one organic C₁-C₆ alkoxysilane (A1) of formula (S-IV) selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,

and/or their condensation products.

The corresponding hydrolysis or condensation products are, for example, the following compounds. Here, the condensation products represent maximally oligomeric compounds, but not polymers.

Hydrolysis of C₁-C₆ alkoxy silane of the formula (S-I) with water (reaction scheme using the example of 3-aminopropyltriethoxysilane):

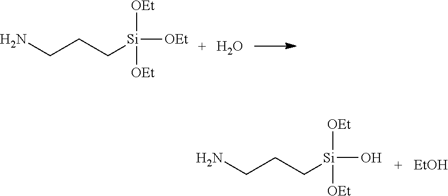 [SMILES: "EtOH" ; CCO[Si](CCCN)(OCC)OCC ; O ; CCO[Si](O)(CCCN)OCC]

Depending on the amount of water used, the hydrolysis reaction can also take place several times per C₁-C₆ alkoxy silane used:

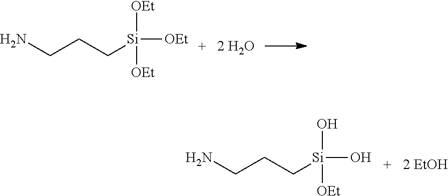 [SMILES: CCO[Si](CCCN)(OCC)OCC ; "2 H2O" ; CCO[Si](O)(O)CCCN ; "2 EtOH"]

respectively

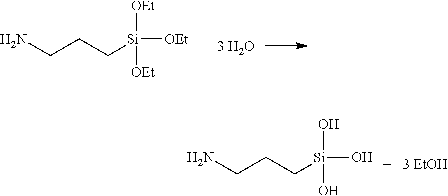 [SMILES: CCO[Si](CCCN)(OCC)OCC ; "3 H2O" ; NCCC[Si](O)(O)O ; "3 EtOH"]

Hydrolysis of C₁-C₆ alkoxy silane of the formula (S-IV) with water (reaction scheme using the example of methyltrimethoxysilane):

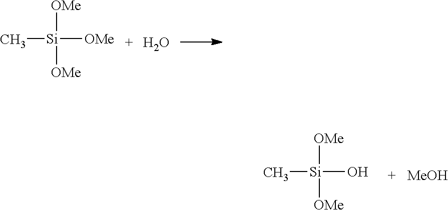

Depending on the amount of water used, the hydrolysis reaction can also take place several times per C₁-C₆ alkoxy silane used:

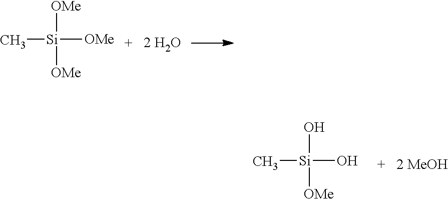

respectively

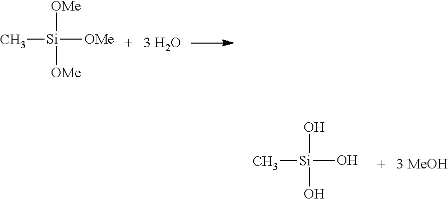

Possible condensation reactions include (shown using the mixture (3-aminopropyl)triethoxysilane and methyltrimethoxysilane):

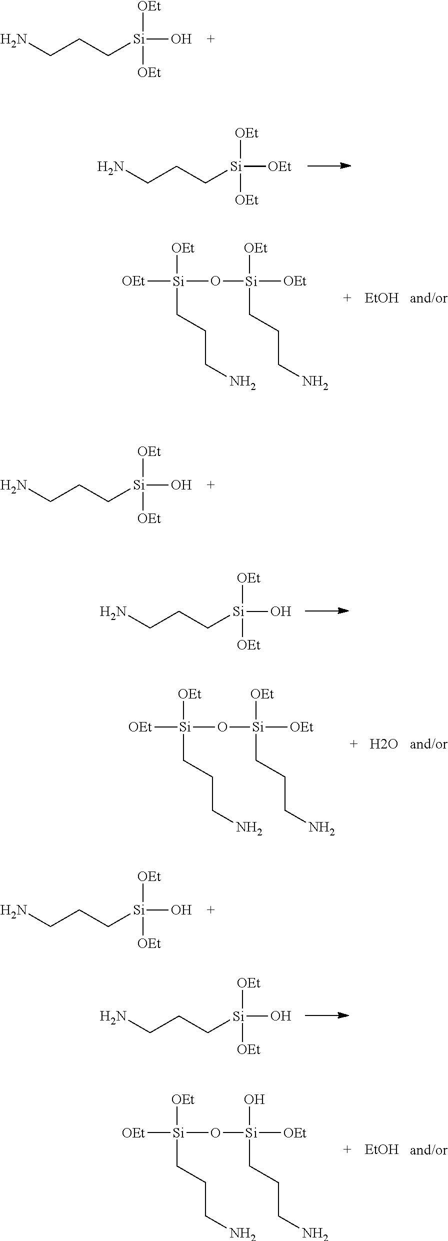

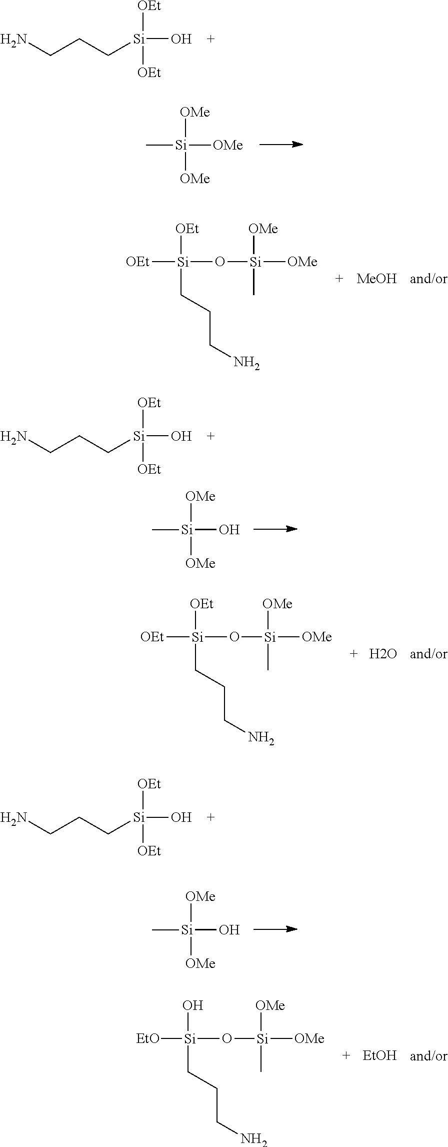

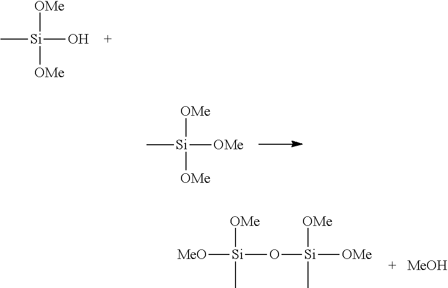

In the above exemplary reaction schemes, condensation to a dimer is shown in each case, but further condensations to oligomers with multiple silane atoms are also possible and preferred too.

Both partially hydrolyzed and fully hydrolyzed C₁-C₆ alkoxysilanes of the formula (S-I) can participate in these condensation reactions, which undergo condensation with as yet unreacted, partially or also fully hydrolyzed C₁-C₆ alkoxysilanes of the formula (S-I). In this case, the C₁-C₆ alkoxysilanes of formula (S-I) react with themselves.

Furthermore, both partially hydrolyzed and fully hydrolyzed C₁-C₆-alkoxysilanes of the formula (S-I) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-I) react with the C₁-C₆ alkoxysilanes of formula (S-IV).

Furthermore, both partially hydrolyzed and fully hydrolyzed C₁-C₆ alkoxysilanes of the formula (S-IV) can also participate in the condensation reactions, which undergo condensation with as yet unreacted, partially or also fully hydrolyzed C₁-C₆ alkoxysilanes of the formula (S-IV). In this case, the C₁-C₆ alkoxysilanes of formula (S-IV) react with themselves.

The composition (A) as contemplated herein may contain one or more organic C₁-C₆ alkoxysilanes (A1) in various proportions. The skilled person determines this depending on the desired thickness of the silane coating on the keratin material and on the amount of keratin material to be treated.

Particularly storage-stable preparations with very good dyeing results in application could be obtained if the composition (A) comprises—based on its total weight—one or more organic C₁-C₆-alkoxysilanes (A1) and/or the condensation products thereof in a total amount of 40.0 to 99.0% by weight of the composition (A) %, preferably from 50.0 to 98.0% by weight, more preferably from 60.0 to 97.0% by weight, still more preferably from 70.0 to 96.0% by weight and most preferably from 80.0 to 95.0% by weight.

In a further embodiment, a very particularly preferred method is described wherein the composition (A) comprises—based on the total weight of the composition (A)—one or more organic C₁-C₆-alkoxysilanes (A1) and/or the condensation products thereof in a total amount of from 40.0 to 99.0 wt %, preferably from 50.0 to 98.0% by weight, more preferably from 60.0 to 97.0% by weight, still more preferably from 70.0 to 96.0% by weight and most preferably from 80.0 to 95.0% by weight.

### Other Cosmetic Ingredients in the Composition (A)

In addition, the composition (A) may also contain one or more other cosmetic ingredients.

The cosmetic ingredients that may be optionally used in the composition (A) may be any suitable ingredients to impart further beneficial properties to the product. For example, in the composition (A), a solvent, a surface-active compound from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

The selection of these further substances will be made by the skilled person according to the desired properties of the agents. With regard to further optional components as well as the quantities of these components used, reference is expressly made to the relevant manuals known to the skilled person.

### Water Content (A1) in the Composition (A)

The method as contemplated herein is exemplified by the application of a first composition (A) on the keratinous material.

To ensure sufficiently high storage stability, composition (A) may be exemplified by being low in water, preferably substantially free of water. Therefore, the composition (A) preferably comprises—based on the total weight of the composition (A)—less than 15% by weight of water.

At a water content of just below 15% by weight, the compositions (A) are stable in storage over longer periods. However, in order to further improve the storage stability and to ensure a sufficiently high reactivity of the organic C₁-C₆ alkoxy silanes (A2), it has been found to be particularly preferable to further lower the water content in the composition (A). For this reason, first composition (A)—based on the total weight of composition (A)—preferably comprises 0.01 to 15.0% by weight, preferably 0.1 to 13.0% by weight, further preferably 0.5 to 11.0% and most preferably 1.0 to 9.0% by weight of water.

In the context of a very particularly preferred embodiment, a method as contemplated herein is described wherein the first composition (A) comprises—based on the total weight of the composition (A)—0.01 to 15.0% by weight, preferably 0.1 to 13.0% by weight, further preferably 0.5 to 11.0 and very particularly preferably 1.0 to 9.0% by weight of water.

However, in a further embodiment, a water-comprising composition (A) can also be applied to the keratin material. In the context of this embodiment, a method as contemplated herein is described wherein the first composition (A) comprises—based on the total weight of the composition (A)—50.0 to 99.0% by weight, preferably 60.0 to 98.0% by weight, further preferably 65.0 to 97.0 and very particularly preferably 70.0 to 96.0% by weight of water.

### pH Value of the Compositions (A)

In further experiments, it has been found that the pH values of composition (A) can have an influence on the color intensities obtained during dyeing. It was found that alkaline pH values in particular have a beneficial effect on the dyeing performance achievable in the method.

For this reason, it is preferred that the compositions (A) have a pH of from 7.0 to 12.0, preferably from 7.5 to 11.5, more preferably from 8.0 to 11.0, and most preferably from 8.0 to 10.5.

The pH value can be measured using the usual methods known from the state of the art, such as pH measurement using glass electrodes via combination electrodes or using pH indicator paper.

In another very particularly preferred embodiment, a method as contemplated herein, is described wherein the composition (A) has a pH of from 7.0 to 12.0, preferably from 7.5 to 11.5, more preferably from 8.0 to 11.0 and most preferably from 8.0 to 10.5.

### Celluloses in the Composition (B)

The method as contemplated herein comprises the application of a second composition (B) on the keratin material. In this case, the composition (B) is described wherein it comprises

a first cellulose (B1) and a second cellulose (B2), the second cellulose (B2) being different from the first cellulose (B1).

For the purposes of this present disclosure, a cellulose means both cellulose itself and a derivative thereof, i.e., a chemically or physically modified cellulose.

Cellulose is composed of 0-1,4-glycosidic-linked D-glucopyranose units. In the solid state, crystalline regions alternate with regions of weak order (amorphous regions) in cellulose. Natural and manufacturing-related impurities, such as the presence of carboxy groups in particular, are typically in the range of about 1%. As contemplated herein, cellulose itself is therefore not considered an anionic polysaccharide.

Cellulose usable as contemplated herein has a degree of polymerization (DP), i.e. a chain length of glucopyranose units, of 10 to about 8000. However, it has been found that celluloses with a low degree of polymerization in particular exert a positive effect on the dyeing properties of the agents.

So-called microcrystalline cellulose in particular shows beneficial effects in this respect. Microcrystalline cellulose is obtained by partial alkaline or acid hydrolysis of celluloses, in which only the amorphous regions of the semicrystalline cellulose are attacked and completely dissolved. This initially results in microfine cellulose, which is disaggregated into microcrystalline cellulose in aqueous suspension under the action of mechanical force.

The degree of polymerization remaining after hydrolysis (also called levelling-off degree of polymerization=LODP) of microcrystalline cellulose is in the range of approx. 30-400.

Preferred celluloses are therefore microcrystalline celluloses and have a degree of polymerization of 30 to 400.

For the purposes of this present disclosure, a cellulose (B1) or (B2) is also understood to be a derivative of a cellulose, i.e. the cellulose can be provided with substituents and/or carry further chemical functional groups by reaction with a chemical agent.

Corresponding chemically modified celluloses (B1) or (B2) can be nonionic, cationic and/or anionic.

A suitable cationic cellulose is marketed, for example, under the name Polymer JR® 400 by Amerchol and has the INCI designation Polyquaternium-10. Another cationic cellulose bears the INCI designation polyquatemium-24 and is sold under the trade name Polymer LM-200 from Amerchol or also Quatrisoft® LM 200. Other commercial products include the compounds Celquat® H 100, Celquat® and L 200. The commercial products mentioned are preferred cationic celluloses.

Quite particularly preferred are the nonionic celluloses. These may be selected, for example, from the group comprising hydroxyethyl cellulose, hydroxypropyl cellulose, hydroxyethyl methyl cellulose, hydroxypropyl methyl cellulose, hydroxybutyl methyl cellulose, hydroxyethyl ethyl cellulose, hydroxypropyl methyl cellulose, methyl ethyl cellulose and ethyl cellulose.

Particularly suitable for solving the problem as contemplated herein are the non-ionic celluloses from the group of cellulose ethers, from the group of hydroxyethyl cellulose, hydroxypropyl cellulose and hydroxypropyl methyl cellulose. These are marketed, for example, under the trademarks Culminal® and Benecel® and Natrosol® grades by the companies Aqualon, Hercules or Ashland.

A hydroxypropyl cellulose with a molecular weight of 30,000 to 50,000 g/mol, which is marketed for example under the trade name Nisso Sl® by Lehmann & Voss, Hamburg, is also particularly suitable.

Suitable anionic celluloses are, for example, carboxymethyl cellulose, carboxymethyl hydroxyethyl cellulose, cellulose acetate, cellulose acetate butyrate, cellulose acetate propionate, cellulose acetate propionate carboxylate and/or their physiologically acceptable salts.

The best results were obtained with the use of non-ionic celluloses. For this reason, it is particularly preferable if the components used in the method as contemplated herein are in a composition (B) comprising

(B1) a first nonionic cellulose and
(B2) a second nonionic cellulose different from the first cellulose (B1).

Within the group of celluloses, especially within the group of nonionic celluloses, celluloses modified with a hydroxy-C₁-C₆-alkyl group have proven to be particularly suitable.

Celluloses carrying at least one 2-hydroxyethyl group, carrying at least one 2-hydoxypropyl group and/or carrying at least one 3-hydroxypropyl group have proved to be particularly suitable for solving the problem as contemplated herein.

In a further embodiment, a very particularly preferred method is described wherein the second composition (B) comprises

(B1) a first cellulose bearing at least one hydroxyethyl group, and
(B2) a second cellulose bearing at least one hydoxypropyl group.

In a further embodiment, a very particularly preferred method is described wherein the second composition (B) comprises

(B1) a first cellulose bearing at least one 2-hydroxyethyl group, and
(B2) a second cellulose bearing at least one 2-hydroxypropyl group and/or one 3-hydroxypropyl group.

The two celluloses (B1) and (B2) are structurally different from each other.

A particularly well-suited cellulose with a 2-hydroxyethyl group is 2-hydroxethylcellulose, CAS No. 9004-62-0, which can be obtained commercially under the trade name Natrosol 250 HR from the company Ashland (Hercules).

A particularly well-suited cellulose with a hydroxypropyl group is hydroxypropyl cellulose, CAS No. 9004-64-2, which can be purchased under the trade name Klucel H CS from the Hercules company.

A particularly suitable cellulose with a hydroxypropyl group is hydroxypropylmethyl cellulose, CAS No. 9004-65-3, which can be purchased under the trade name Benecel K 4 M from Ashland (or Hercules) or under the trade name Methocel 267 from Dow.

To optimize consistency and applicability, the celluloses (B1) and (B2), in particular the aforementioned preferred and especially preferred representatives, are preferably used in certain quantity ranges in the composition (B).

Thus, it is particularly preferred if the composition (B)—based on the total weight of the composition (B)—comprises

(B1) comprises 0.1 to 10.0% by weight, preferably 0.1 to 8.0% by weight, more preferably 0.1 to 6.0% by weight and very particularly preferably 0.1 to 4.0% by weight of 2-hydroxyethyl cellulose.

In a further embodiment, a very particularly preferred method is described wherein the second composition (B)—based on the total weight of the composition (B)—comprises

(B1) 0.1 to 10.0% by weight, preferably 0.1 to 8.0% by weight, more preferably 0.1 to 6.0% by weight and most preferably 0.1 to 4.0% by weight of 2-hydroxyethyl cellulose.

Furthermore, it is particularly preferred if the composition (B)—based on the total weight of the composition (B)—comprises

(B2) comprises one or more celluloses selected from the group of 2-hydroxypropyl cellulose, 3-hydroxypropyl cellulose, 2-hydroxypropyl methyl cellulose and/or 3-hydroxypropyl methyl cellulose in a total amount of 0.1 to 8.0% by weight, more preferably 0.1 to 6.0% by weight and most preferably 0.1 to 4.0% by weight.

In a further embodiment, a very particularly preferred method is described wherein the second composition (B)—based on the total weight of the composition (B)—comprises (B2) one or more celluloses selected from the group of 2-hydroxypropyl cellulose, 3-hydroxypropyl cellulose, 2-hydroxypropyl methyl cellulose and/or 3-hydroxypropyl methyl cellulose in a total amount of 0.1 to 8.0% by weight, more preferably 0.1 to 6.0% by weight and most preferably 0.1 to 4.0% by weight.

In addition, particularly good effects were obtained when celluloses (B1) and (B2) were used in certain weight ratios to each other in composition (B).

In a further embodiment, a very particularly preferred method is described wherein the weight ratio of all celluloses (B1) included in the composition (B) to all celluloses (B2) included in the composition (B), i.e. the weight ratio (B1)/(B2), is at a value of from 0.2 to 5.0 preferably from 0.3 to 3.0, more preferably from 0.5 to 2.0 and most preferably from 0.8 to 1.5.

In a further embodiment, a very particularly preferred method is described wherein the weight ratio of all hydroxyethyl celluloses (B1) included in the composition (B) to all hydroxypropyl celluloses, (B2) included in the composition (B), i.e. the weight ratio (B1)/(B2), is at a value of from 0.2 to 5.0 preferably from 0.3 to 3.0, more preferably from 0.5 to 2.0 and most preferably from 0.8 to 1.5.

### Water Content of the Composition (B)

Composition (B) comprises celluloses (B1) and (B2) in a cosmetic carrier, preferably in an aqueous cosmetic carrier.

In this context, it has been found to be preferred if the composition (B) comprises—based on the total weight of the composition (B)—5.0 to 90.0% by weight, preferably 30.0 to 98.0% by weight, more preferably 40.0 to 95.0% by weight, further preferably 45.0 to 90.0% by weight, still more preferably 50.0 to 90.0% by weight and most preferably 55.0 to 90.0% by weight of water.

In the context of a further embodiment, a method as contemplated herein is described wherein the second composition (B) comprises—based on the total weight of the composition (B)—30.0 to 98.0% by weight, preferably 40.0 to 95.0% by weight, more preferably 45.0 to 90.0% by weight, still more preferably 50.0 to 90.0% by weight and most preferably 55.0 to 90.0% by weight of water.

### Other Cosmetic Ingredients in the Composition (B)

In addition, the composition (B) may also contain one or more further cosmetic ingredients.

The cosmetic ingredients that may be optionally used in the composition (B) may be any suitable ingredients to impart further beneficial properties to the product. For example, a solvent, a surface-active compound from the group of nonionic, cationic, anionic or zwitterionic/amphoteric surfactants, coloring compounds from the group of pigments, direct dyes, film-forming polymers, fatty components from the group of C₈-C₃₀ fatty alcohols, hydrocarbon compounds, fatty acid esters, acids and bases belonging to the group of pH regulators, perfumes, preservatives and plant extracts.

The selection of these further substances will be made by the skilled person according to the desired properties of the agents. With regard to further optional components as well as the quantities of these components used, reference is expressly made to the relevant manuals known to the skilled person.

### Use of Other Colorant Compounds

In the course of the work leading to the present disclosure, it was observed that the films formed on the keratin material possessed not only good rub fastness but also particularly high color intensity when a coloring compound from the group of pigments and/or direct dyes was used in the method. The use of pigments has proved to be particularly preferable. These additional coloring compounds can be incorporated into composition (A) and/or composition (B).

In another particularly preferred embodiment, a method as contemplated herein is described wherein the first composition (A) comprises at least one colorant compound selected from the group of pigments and/or direct dyes.

In another particularly preferred embodiment, a method as contemplated herein is described wherein the second composition (B) comprises at least one colorant compound selected from the group of pigments and/or direct dyes.

Furthermore, it is also possible to incorporate the colorant compounds into a third, separately prepared composition (C), which is then applied to the keratinous material.

In a further embodiment, the preferred method is one in which the keratinous material is coated with a keratinous material:
a third composition (C) comprising at least one colorant compound selected from the group of pigments and/or direct dyes.

The colorant compound(s) may be selected from the group of pigments and direct dyes, where direct dyes may also be photochromic dyes and thermochromic dyes.

Very preferably, the composition (A) and/or the composition (B) and/or the optionally applicable composition (C) comprises at least one pigment.

According to the present disclosure, pigments are colorant compounds which have a solubility in water at 25° C. of less than 0.5 g/L, preferably less than 0.1 g/L, still more preferably less than 0.05 g/L. Water solubility, for example, can be done using the method described below: 0.5 g of the pigment is weighed out in a beaker. A stirring bar is added. Then one liter of distilled water is added. This mixture is heated to 25° C. for one hour with stirring on a magnetic stirrer. If undissolved components of the pigment are still visible in the mixture after this period, the solubility of the pigment is below 0.5 g/L. If the pigment-water mixture cannot be visually assessed due to the high intensity of the pigment, which may be finely dispersed, the mixture is filtered. If a portion of undissolved pigment remains on the filter paper, the solubility of the pigment is below 0.5 g/L.

Suitable color pigments can be of inorganic and/or organic origin.

In a preferred embodiment, a composition as contemplated herein is described wherein it comprises at least one colorant compound selected from the group of inorganic and/or organic pigments.

Preferred color pigments are selected from synthetic or natural inorganic pigments. Inorganic color pigments of natural origin can be produced, for example, from chalk, ocher, umber, green earth, burnt terra di Siena or graphite. Furthermore, black pigments such as iron oxide black, colored pigments such as ultramarine or iron oxide red, and fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulfates, chromates and/or molybdates. Particularly preferred color pigments are black iron oxide (CI 77499), yellow iron oxide (CI 77492), red and brown iron oxide (CI 77491), manganese violet (CI 77742), ultramarines (sodium aluminum sulfosilicates, CI 77007, Pigment Blue 29), chromium oxide hydrate (CI77289), iron blue (ferric ferrocyanide, CI77510) and/or carmine (cochineal).

Colored pearlescent pigments are also particularly preferred colorants from the group of pigments as contemplated herein. These are usually mica and/or mica-based and may be coated with one or more metal oxides. Mica belongs to the layer silicates. The main representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

In a very particularly preferred embodiment, a method as contemplated herein is described wherein the composition (A) and/or the composition (B) comprises at least one colorant compound from the group of inorganic pigments selected from the group of colored metal oxides, metal hydroxides, metal oxide hydrates, silicates, metal sulfides, complex metal cyanides, metal sulfates, bronze pigments and/or colored mica- or mica-based pigments coated with at least one metal oxide and/or a metal oxychloride.

As an alternative to natural mica, synthetic mica coated with one or more metal oxides can also be used as a pearlescent pigment. Particularly preferred pearlescent pigments are based on natural or synthetic mica and are coated with one or more of the metal oxides mentioned above. The color of the respective pigments can be varied by varying the layer thickness of the metal oxide(s).

In a further preferred embodiment, the composition (A) as contemplated herein and/or the composition (B) is described wherein it comprises at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition (A) and/or composition (B) as contemplated herein is described wherein it comprises at least one colorant compound selected from mica- or mica-based pigments which are reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Very particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aboriginal Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Further particularly preferred color pigments with the trade name Xirona® are, for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are, for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

Timiron Synwhite Satin, Merck, Synthetic Fluorphlogopite, Titanium Dioxide, Tin Oxide

### Timiron Super Blue, Merck, Mica, CI 77891 (Titanium Dioxide)

Timiron Diamond Cluster MP 149, Merck, Mica, CI 77891 (Titan dioxide)
Timiron Splendid Gold, Merck, CI 77891 (Titanium dioxide), Mica, Silica
Timiron Super Sulver, Merck, Mica, CI 77891 (Titanium dioxide)

Within the scope of a further embodiment, the composition (A) and/or the composition (B) and/or an optionally usable composition (C) may also comprise one or more color-imparting compounds from the group of organic pigments

The organic pigments of the present disclosure are correspondingly insoluble organic dyes or colorants which may be selected, for example, from the group comprising nitroso-, nitro-azo-, xanthene-, anthraquinone-, isoindolinone-, isoindoline-, quinacridone-, perinone-, perylene-, diketo-pyrrolopyorrole-, indigo-, thioindido-, dioxazine-, and/or triarylmethane compounds.

Particularly suitable organic pigments are, for example, carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a method as contemplated herein is described wherein the composition (A) and/or the composition (B) comprises at least one colorant compound from the group of organic pigments selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a colored varnish. As contemplated herein, the term color varnish means particles comprising a layer of absorbed dyes, the unit of particle and dye being insoluble under the above conditions. The particles may be, for example, inorganic substrates, which may be aluminum, silica, calcium borosilicate, calcium aluminum borosilicate, or aluminum.

Alizarin color varnish, for example, can be used as a color varnish.

Due to their excellent light and temperature resistance, the use of the above pigments in the composition as contemplated herein is particularly preferred. Furthermore, it is preferred if the pigments used have a certain particle size. On the one hand, this particle size leads to an even distribution of the pigments in the polymer film formed and, on the other hand, avoids a rough hair or skin feeling after application of the cosmetic product. It is therefore advantageous as contemplated herein if the at least one pigment has a mean particle size D50 of from 1.0 to 50 μm, preferably from 5.0 to 45 μm, preferably from 10 to 40 μm, in particular from 14 to 30 μm. For example, the mean particle size D₅₀ can be determined using dynamic light scattering (DLS).

Pigments with a specific shaping may also have been used to color the keratin material. For example, a pigment based on a lamellar and/or a lenticular substrate platelet can be used. Furthermore, coloring based on a substrate platelet comprising a vacuum metallized pigment is also possible.

In a further embodiment, the composition (A) and/or the composition (B) and/or an optionally applicable composition (C) may also comprise one or more colorant compounds selected from the group of lamellar substrate platelet-based pigments, lenticular substrate platelet-based pigments and vacuum metallized pigments.

The substrate platelets of this type have an average thickness of at most 50 nm, preferably less than 30 nm, particularly preferably at most 25 nm, for example at most 20 nm. The average thickness of the substrate platelets is at least 1 nm, preferably at least 2.5 nm, further preferably at least 5 nm, for example at least 10 nm. Preferred ranges for substrate platelet thickness are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm, and 10 to 20 nm. Preferably, each substrate plate has a thickness that is as uniform as possible.

Due to the low thickness of the substrate platelets, the pigment exhibits particularly high hiding power.

The substrate platelets have a monolithic structure. Monolithic in this context means comprising a single self-included unit without fractures, stratifications or inclusions, although structural changes may occur within the substrate platelets. The substrate platelets are preferably homogeneous in structure, i.e. no concentration gradient occurs within the platelets. In particular, the substrate platelets are not layered and do not have particles or particulates distributed therein.

The size of the substrate platelet can be adjusted to the respective application purpose, in particular the desired effect on the keratinous material. Typically, the substrate platelets have an average largest diameter of about 2 to 200 μm, especially about 5 to 100 μm.

In a preferred embodiment, the shape factor (aspect ratio), expressed by the ratio of the average size to the average thickness, is at least 80, preferably at least 200, more preferably at least 500, especially preferably more than 750. Here, the average size of the uncoated substrate platelets means the d50 value of the uncoated substrate platelets. Unless otherwise stated, the d50 value was determined using a Sympatec Helos instrument with Quixel wet dispersion. For sample preparation, the sample to be analyzed was predispersed in isopropanol for a period of 3 minutes.

The substrate platelets can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-)precious stones, and plastics. Preferably, the substrate platelets are constructed of metal (alloy).

Any metal suitable for metallic luster pigments can be used. Such metals include iron and steel, as well as all air- and water-resistant (semi)metals such as platinum, zinc, chromium, molybdenum and silicon, as well as their alloys such as aluminum bronzes and brass. Preferred metals are aluminum, copper, silver and gold. Preferred substrate platelets include aluminum platelets and brass platelets, with aluminum substrate platelets being particularly preferred.

Lamellar substrate platelets are described wherein an irregularly structured edge and are also referred to as “cornflakes” due to their appearance.

Due to their irregular structure, pigments based on lamellar substrate platelets generate a high proportion of scattered light. In addition, pigments based on lamellar substrate platelets do not completely cover the existing color of a keratinous material, and effects analogous to natural graying can be achieved, for example.

Lenticular (=lens-shaped) substrate platelets have an essentially regular round edge and are also called “silverdollars” due to their appearance. Due to their regular structure, pigments based on lenticular substrate platelets have a predominance of reflected light.

Vacuum metallized pigments (VMP) can be obtained, for example, by releasing metals, metal alloys or metal oxides from suitably coated films. They are exemplified by a particularly low thickness of the substrate platelets in the range of 5 to 50 nm and by a particularly smooth surface with increased reflectivity. Substrate platelets comprising a vacuum metallized pigment are also referred to as VMP substrate platelets in the context of this application. VMP substrate platelets made of aluminum can be obtained, for example, by releasing aluminum from metallized foils.

The metal or metal alloy substrate platelets can be passivated, for example by anodizing (oxide layer) or chromating.

Uncoated lamellar, lenticular, and/or VPM substrate platelets, especially those made of metal or metal alloy, reflect incident light to a high degree and produce a light-dark flop but no color impression.

A color impression can be created, for example, due to optical interference effects. Such pigments may be based on at least single-coated substrate platelets. These show interference effects by superposition of differently refracted and reflected light rays.

Accordingly, preferred pigments are those based on a coated lamellar substrate platelet. The substrate platelet preferably has at least one coating B of a highly refractive metal oxide having a coating thickness of at least 50 nm. There is preferably another coating A between the coating B and the surface of the substrate platelet. If necessary, there is a further coating C on the layer B, which is different from the layer B underneath.

Suitable materials for coatings A, B and C are all substances that can be applied to the substrate platelets in a film-like and permanent manner and, in the case of coatings A and B, have the required optical properties. Generally, coating part of the surface of the substrate platelets is sufficient to obtain a pigment with a glossy effect. For example, only the top and/or bottom of the substrate platelets may be coated, with the side surface(s) omitted. Preferably, the entire surface of the optionally passivated substrate platelets, including the side surfaces, is covered by coating B. The substrate platelets are thus completely enveloped by coating B. This improves the optical properties of the pigment and increases its mechanical and chemical resistance. The above also applies to layer A and preferably also to layer C, if present.

Although multiple coatings A, B and/or C may be present in each case, the coated substrate platelets preferably have only one coating A, B and, if present, C in each case.

The coating B is composed of at least one highly refractive metal oxide. Highly refractive materials have a refractive index of at least 1.9, preferably at least 2.0, and more preferably at least 2.4. Preferably, the coating B comprises at least 95 wt %, more preferably at least 99 wt %, of high refractive index metal oxide(s).

The coating B has a thickness of at least 50 nm. Preferably, the thickness of coating B is no more than 400 nm, more preferably no more than 300 nm.

Highly refractive metal oxides suitable for coating B are preferably selectively light-absorbing (i.e. colored) metal oxides, such as iron(III) oxide (α- and γ-Fe2O3, red), cobalt (II) oxide (blue), chromium (III) oxide (green), titanium (III) oxide (blue, usually present in admixture with titanium oxynitrides and titanium nitrides) and vanadium (V) oxide (orange), and mixtures thereof. Colorless high-index oxides such as titanium dioxide and/or zirconium oxide are also suitable.

Coating B may contain a selectively absorbing dye, preferably 0.001 to 5% by weight, particularly preferably 0.01 to 1% by weight, in each case based on the total amount of coating B. Suitable dyes are organic and inorganic dyes which can be stably incorporated into a metal oxide coating.

The coating A preferably has at least one low refractive index metal oxide and/or metal oxide hydrate. Preferably, coating A comprises at least 95 wt %, more preferably at least 99 wt %, of low refractive index metal oxide (hydrate). Low refractive index materials have a refractive index of 1.8 or less, preferably 1.6 or less.

Low refractive index metal oxides suitable for coating A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. The coating A preferably has a thickness of 1 to 100 nm, further preferably 5 to 50 nm, especially preferably 5 to 20 nm.

Preferably, the distance between the surface of the substrate platelets and the inner surface of coating B is at most 100 nm, particularly preferably at most 50 nm, especially preferably at most 20 nm. By ensuring that the thickness of coating A, and thus the distance between the surface of the substrate platelets and coating B, is within the range specified above, it is possible to ensure that the pigments have a high hiding power.

If the pigment based on a lamellar substrate platelet has only one layer A, it is preferred that the pigment has a lamellar substrate platelet of aluminum and a layer A of silica. If the pigment based on a lamellar substrate platelet has a layer A and a layer B, it is preferred that the pigment has a lamellar substrate platelet of aluminum, a layer A of silica and a layer B of iron oxide.

According to a preferred embodiment, the pigments have a further coating C of a metal oxide (hydrate), which is different from the underlying coating B. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Preferred is silicon dioxide.

The coating C preferably has a thickness of 10 to 500 nm, more preferably 50 to 300 nm. By providing coating C, for example based on TiO₂, better interference can be achieved while maintaining high hiding power.

Layers A and C serve in particular as corrosion protection as well as chemical and physical stabilization. Particularly preferred layers A and C are silica or alumina applied by the sol-gel process. This method comprises dispersing the uncoated lamellar substrate platelets or the lamellar substrate platelets already coated with layer A and/or layer B in a solution of a metal alkoxide such as tetraethyl orthosilicate or aluminum triisopropanolate (usually in a solution of organic solvent or a mixture of organic solvent and water with at least 50% by weight of organic solvent such as a C1 to C4 alcohol), and adding a weak base or acid to hydrolyze the metal alkoxide % organic solvent such as a C1 to C4 alcohol), and adding a weak base or acid to hydrolyze the metal alkoxide, thereby forming a film of the metal oxide on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-comprising layer to the oxide layers by annealing).

Although each of the coatings A, B and/or C may be composed of a mixture of two or more metal oxide(hydrate)s, each of the coatings is preferably composed of one metal oxide(hydrate).

The pigments based on coated lamellar or lenticular substrate platelets or the pigments based on coated VMP substrate platelets preferably have a thickness of 70 to 500 nm, particularly preferably 100 to 400 nm, especially preferably 150 to 320 nm, for example 180 to 290 nm. Due to the low thickness of the substrate platelets, the pigment exhibits particularly high hiding power. The low thickness of the coated substrate platelets is achieved in particular by keeping the thickness of the uncoated substrate platelets low, but also by adjusting the thicknesses of the coatings A and, if present, C to as small a value as possible. The thickness of coating B determines the color impression of the pigment.

The adhesion and abrasion resistance of pigments based on coated substrate platelets in keratinic material can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-comprising, layer A, B, or C. The outermost layer denotes the layer that is spatially farthest from the lamellar substrate platelet. The organic compounds are preferably functional silane compounds that can bind to the metal oxide-comprising layer A, B, or C. These can be either mono- or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyl-triethoxysilane, 2-acryloxyethyltriethoxysilane, 3-methacryloxypropyltris(methox-yethoxy)silane, 3-methacryloxypropyltris(butoxyethoxy)silane, 3-methacryloxy-propyltris(propoxy)silane, 3-methacryloxypropyltris(butoxy)silane, 3-acryloxy-propyltris(methoxyethoxy)silane, 3-acryloxypropyltris(butoxyethoxy)silane, 3-acryl-oxypropyltris(butoxy)silane, vinyltrimethoxysilane, Vinyltriethoxysilane, vinylethyl dichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, in particular an alkylsilane or arylsilane, can be carried out. This has only one functional group, which can covalently bond to the surface pigment based on coated lamellar substrate platelets (i.e. to the outermost metal oxide-comprising layer) or, if not completely covered, to the metal surface. The hydrocarbon residue of the silane points away from the pigment. Depending on the type and nature of the hydrocarbon residue of the silane, a different degree of hydrophobicity of the pigment is achieved. Examples of such silanes include hexadecyltrimethoxysilane, propyltrimethoxysilane, etc. Particularly preferred are pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

Suitable pigments based on a lamellar substrate platelet include, for example, the pigments of the VISIONAIRE series from Eckart.

Pigments based on a lenticular substrate platelet are available, for example, under the name Alegrace® Gorgeous from the company Schlenk Metallic Pigments GmbH.

Pigments based on a substrate platelet comprising a vacuum metallized pigment are available, for example, under the name Alegrace® Marvelous or Alegrace® Aurous from the company Schlenk Metallic Pigments GmbH.

In a further embodiment, a method as contemplated herein is described wherein the composition (A) comprises—based on the total weight of the composition (A)—one or more pigments in a total amount of from 0.001 to 20% by weight, in particular from 0.05 to 5% by weight.

In a further embodiment, a method as contemplated herein is described wherein the composition (B) comprises—based on the total weight of the composition (B)—one or more pigments in a total amount of from 0.001 to 20% by weight, in particular from 0.05 to 5% by weight.

As coloring compounds, the compositions as contemplated herein may also contain one or more direct dyes. Direct-acting dyes are dyes that are absorbed directly into the hair and do not require an oxidative process to form the color. Direct dyes are usually nitrophenylenediamines, nitroaminophenols, azo dyes, anthraquinones, triarylmethane dyes or indophenols.

The direct dyes according to the present disclosure have a solubility in water (760 mmHg) at 25° C. of more than 0.5 g/L and are therefore not to be regarded as pigments. Preferably, the direct dyes according to the present disclosure have a solubility in water (760 mmHg) at 25° C. of more than 1.0 g/L. Particularly preferably, the direct dyes according to the present disclosure have a solubility in water (760 mmHg) at 25° C. of greater than 1.5 g/L.

Direct dyes can be divided into anionic, cationic and nonionic direct dyes.

In a further preferred embodiment, an agent as contemplated herein is described wherein it comprises at least one anionic, cationic and/or nonionic direct dye as the coloring compound.

In a further preferred embodiment, a method as contemplated herein is described wherein the composition (B) and/or the composition (C) comprises at least one colorant compound selected from the group of anionic, nonionic, and/or cationic direct dyes.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

Examples of nonionic direct dyes that can be used are nonionic nitro and quinone dyes and neutral azo dyes. Suitable nonionic direct dyes are those available under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid, and 2-chloro-6-ethylamino-4-nitrophenol.

Anionic direct dyes are also called acid dyes. Acid dyes are direct dyes that have at least one carboxylic acid moiety (—COOH) and/or one sulfonic acid moiety (—SO₃H). Depending on the pH, the protonated forms (—COOH, —SO₃H) of the carboxylic or sulfonic acid moieties are in equilibrium with their deprotonated forms (—COO⁻, —SO₃⁻ present). As pH decreases, the proportion of protonated forms increases. If direct dyes are used in the form of their salts, the carboxylic acid groups or sulfonic acid groups are present in deprotonated form and are neutralized with corresponding stoichiometric equivalents of cations to maintain electroneutrality. Acid dyes as contemplated herein can also be used in the form of their sodium salts and/or their potassium salts.

The acid dyes according to the present disclosure have a solubility in water (760 mmHg) at 25° C. of more than 0.5 g/L and are therefore not to be regarded as pigments. Preferably, the acid dyes according to the present disclosure have a solubility in water (760 mmHg) at 25° C. of more than 1.0 g/L.

The alkaline earth salts (such as calcium salts and magnesium salts) or aluminum salts of acid dyes often have poorer solubility than the corresponding alkali salts. If the solubility of these salts is below 0.5 g/L (25° C., 760 mmHg), they do not fall under the definition of a direct dye.

A key feature of acid dyes is their ability to form anionic charges, so the carboxylic or sulfonic acid groups responsible for this are usually linked to various chromophoric systems. Suitable chromophoric systems are found, for example, in the structures of nitrophenylenediamines, nitroaminophenols, azo dyes, anthraquinone dyes, triarylmethane dyes, xanthene dyes, rhodamine dyes, oxazine dyes, and/or indophenol dyes.

For example, one or more compounds from the following group can be selected as particularly well-suited acid dyes: Acid Yellow 1 (D&C Yellow 7, Citronin A, Ext. D&C Yellow No. 7, Japan Yellow 403, CI 10316, COLIPA n^{o} B001), Acid Yellow 3 (COLIPA n°: C 54, D&C Yellow N^{o} 10, Quinoline Yellow, E104, Food Yellow 13), Acid Yellow 9 (CI 13015), Acid Yellow 17 (CI 18965), Acid Yellow 23 (COLIPA n^{o} C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-naphthol orange, Orange II, CI 15510, D&C Orange 4, COLIPA n^{o} C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C Red 46, True Red D, FD&C Red No. 2, Food Red 9, Naphthol Red S), Acid Red 33 (Red 33, Fuchsia Red, D&C Red 33, CI 17200), Acid Red 35 (CI C.I. 18065), Acid Red 51 (CI 45430, Pyrosine B, Tetraiodofluorescein, Eosin J, Iodeosin), Acid Red 52 (CI 45100, Food Red 106, Solar Rhodamine B, Acid Rhodamine B, Red n^{o} 106 Pontacyl Brilliant Pink), Acid Red 73 (CI 27290), Acid Red 87 (Eosin, CI 45380), Acid Red 92 (COLIPA n^{o} C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet n^{o} 2, C.I. 60730, COLIPA n^{o} C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n^{o} 401, Naphthalene Black 10B, Amido Black 10B, CI 20 470, COLIPA n^{o} B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The water solubility of anionic direct dyes can be determined, for example, in the following way. 0.1 g of the anionic direct dye is added to a beaker. A stirring bar is added. Then 100 ml of water is added. This mixture is heated to 25° C. on a magnetic stirrer while stirring. It is stirred for 60 minutes. The aqueous mixture is then visually assessed. If undissolved residues are still present, the amount of water is increased—for example in steps of 10 ml. Water is added until the amount of dye used has completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml of water at 25° C., the solubility of the dye is 1.0 g/L.

Acid Yellow 1 is named 8-hydroxy-5,7-dinitro-2-naphthalenesulfonic acid disodium salt and has a solubility in water of at least 40 g/L (25° C.).

Acid Yellow 3 is a mixture of the sodium salts of monosulfonic and sisulfonic acids of 2-(2-quinolyl)-1H-indene-1,3(2H)-dione and has a water solubility of 20 g/L (25° C.).

Acid Yellow 9 is the disodium salt of 8-hydroxy-5,7-dinitro-2-naphthalenesulfonic acid, and its water solubility is above 40 g/L (25° C.).

Acid Yellow 23 is the trisodium salt of 4,5-dihydro-5-oxo-1-(4-sulfophenyl)-4-((4-sulfophenyl)azo)-1H-pyrazole-3-carboxylic acid and readily soluble in water at 25° C.

Acid Orange 7 is the sodium salt of 4-[(2-hydroxy-1-naphthyl)azo]benzenesulfonate. Its solubility in water is more than 7 g/L (25° C.).

Acid Red 18 is the trinatrium salt of 7-hydroxy-8-[(E)-(4-sulfonato-1-naphthyl)-diazenyl)]-1,3-naphthalenedisulfonate and has a very high water solubility of more than 20% by weight.

Acid Red 33 is the dinatrium salt of 5-amino-4-hydroxy-3-(phenylazo)-naphthalene-2,7-disulphonate, its solubility in water is 2.5 g/L (25° C.).

Acid Red 92 is the disodium salt of 3,4,5,6-tetrachloro-2-(1,4,5,8-tetrabromo-6-hydroxy-3-oxoxanthen-9-yl)benzoic acid, whose solubility in water is reported to be greater than 10 g/L (25° C.).

Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a water solubility greater than 20% by weight (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to reversibly or irreversibly change its color depending on irradiation with light, especially UV light. This can be done by changing both the intensity and/or the wavelength maximum.

### Film-Forming Polymers

To increase color fastness, the composition (A), the composition (B) and/or the optionally applicable composition (C) may also each contain at least one film-forming polymer.

In the context of a further embodiment, a method as contemplated herein is described wherein the composition (A), the composition (B) and/or the composition (C) comprises at least one film-forming polymer.

Polymers are understood to be macromolecules with a molecular weight of at least 1000 g/mol, preferably of at least 2500 g/mol, further preferably of at least 5000 g/mol, which include identical, repeating organic units. The polymers of the present disclosure may be synthetically produced polymers prepared by polymerizing one type of monomer or by polymerizing different types of monomers that are structurally different from each other. If the polymer is produced by polymerization of a monomer type, it is referred to as homo-polymers. If structurally different monomer types are used in the polymerization, the resulting polymer is called a copolymer.

The maximum molecular weight of the polymer depends on the degree of polymerization (number of polymerized monomers) and the batch size, and is partly determined by the polymerization method. In terms of the present disclosure, it is preferred if the maximum molecular weight of the film-forming hydrophobic polymer (c) is not more than 10⁷ g/mol, preferably not more than 10⁶ g/mol, and further preferably not more than 10⁵ g/mol.

For the purposes of the present disclosure, a film-forming polymer means a polymer capable of forming a film on a substrate, for example on a keratinous material or fiber. The formation of a film can be demonstrated, for example, by viewing the polymer-treated keratin material under a microscope.

The film-forming polymers can be hydrophilic or hydrophobic.

In a first embodiment, it may be preferred to use in the composition (B), at least one hydrophobic film-forming polymer.

A hydrophobic polymer is defined as a polymer that has a solubility in water at 25° C. (760 mmHg) of less than 1% by weight.

For example, the water solubility of the film-forming hydrophobic polymer can be determined in the following way. 1.0 g of the polymer is added to a beaker. Make up to 100 g with water. A stirring bar is added and the mixture is heated to 25° C. on a magnetic stirrer with stirring. It is stirred for 60 minutes. The aqueous mixture is then visually assessed. If the polymer-water mixture cannot be visually assessed due to high turbidity of the mixture, the mixture is filtered. If a portion of undissolved polymer remains on the filter paper, then the solubility of the polymer is less than 1% by weight.

In particular, the polymers of the acrylic acid type, the polyurethanes, the polyesters, the polyamides, the polyureas, the nitrocellulose polymers, the silicone polymers, the polymers of the acrylamide type and the polyisoprenes can be mentioned here.

Particularly suitable film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

Film-forming hydrophobic polymers selected from the group of synthetic polymers, polymers obtainable by free-radical polymerization or natural polymers have proved particularly suitable for solving the problem as contemplated herein.

Other particularly well-suited film-forming hydrophobic polymers can be selected from the homopolymers or copolymers of olefins, such as cycloolefins, butadiene, isoprene or styrene, vinyl ethers, vinyl amides, the esters or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate; isononyl (meth)acrylate; 2-ethylhexyl (meth)acrylate; lauryl (meth)acrylate); isopentyl (meth)acrylate; n-butyl (meth)acrylate); Isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate; and/or mixtures thereof.

Further film-forming hydrophobic polymers may be selected from the homo- or copolymers of (meth)acrylamide; N-alkyl-(meth)acrylamides, especially those with C2-C18 alkyl groups, such as N-ethyl-acrylamide, N-tert-butyl-acrylamide, le N-octyl-crylamide; N-di(C1-C4)alkyl-(meth)acrylamide.

Other preferred anionic copolymers are, for example, copolymers of acrylic acid, methacrylic acid or their C₁-C₆ alkyl esters, as sold under the INCI declaration Acrylates Copolymers. A suitable commercial product is, for example, Aculyn® 33 from Rohm & Haas. However, copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are in particular acrylic acid, methacrylic acid and itaconic acid; suitable alkoxylated fatty alcohols are in particular steareth-20 or ceteth-20.

Very particularly preferred polymers on the market are, for example, Aculyn® 22 (Acrylates/Steareth-20 Methacrylate Copolymer), Aculyn® 28 (Acrylates/Beheneth-25 Methacrylate Copolymer), Structure 2001@ (Acryla-tes/Steareth-20 Itaconate Copolymer), Structure 3001@ (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohme und Haas distributed Soltex OPT (Acrylates/C12-22 Alkyl methacrylate Copolymer).

Suitable polymers based on vinyl monomers may include, for example, the homopolymers and copolymers of N-vinylpyrrolidone, vinylcaprolactam, vinyl-(C1-C6)alkyl-pyrrole, vinyl-oxazole, vinyl-thiazole, vinylpyrimidine, vinylimidazole.

Also particularly suitable are the copolymers octylacrylamide/acrylates/butylaminoethyl methacrylate copolymer, such as those sold commercially under the trade names AMPHOMER® or LOVOCRYL® 47 from NATIONAL STARCH, or the copolymers of acrylates/octylacrylamides sold under the trade names DERMACRYL® LT and DERMACRYL® 79 from NATIONAL STARCH.

Suitable polymers based on olefins include, for example, the homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers may be copolymers comprising one or more blocks in addition to a styrene block, such as styrene/ethylene, styrene/ethylene/butylene, styrene/butylene, styrene/isoprene, styrene/butadiene. Corresponding polymers are sold commercially by BASF under the trade name “Luvitol HSB”.

In a first embodiment, it may be preferred to use at least one hydrophilic film-forming polymer in composition (A), (B) and/or (C).

By a hydrophilic polymer is meant a polymer that has a solubility in water at 25° C. (760 mmHg) of more than 1% by weight, preferably more than 2% by weight.

The water solubility of the film-forming hydrophilic polymer can be determined, for example, in the following way. 1.0 g of the polymer is added to a beaker. Make up to 100 g with water. A stirring bar is added and the mixture is heated to 25° C. on a magnetic stirrer with stirring. It is stirred for 60 minutes. The aqueous mixture is then visually assessed. A completely dissolved polymer appears homogeneous macroscopically. If the polymer-water mixture cannot be visually assessed due to high turbidity of the mixture, the mixture is filtered. If no undissolved polymer remains on the filter paper, then the solubility of the polymer is greater than 1% by weight.

Nonionic, anionic and cationic polymers can be used as film-forming, hydrophilic polymers.

Suitable film-forming, hydrophilic polymers can be selected, for example, from the group of polyvinylpyrrolidone (co)polymers, polyvinyl alcohol (co)polymers, vinyl acetate (co)polymers, the carboxyvinyl (co)polymers, the acrylic acid (co)polymers, the methacrylic acid (co)polymers, the natural gums, the polysaccharides and/or the acrylamide (co)polymers.

Furthermore, it is particularly preferred to use polyvinylpyrrolidone (PVP) and/or a vinylpyrrolidone-comprising copolymer as the film-forming hydrophilic polymer.

It is further preferred if the composition (A), (B) and/or (C) as contemplated herein comprises polyvinylpyrrolidone (PVP) as the film-forming hydrophilic polymer. Surprisingly, the color fastness of the dyeings obtained with agents comprising PVP was also very good.

Particularly well-suited polyvinylpyrrolidones are available, for example, under the name Luviskol® K from BASF SE, especially Luviskol® K 90 or Luviskol®K 85 from BASF SE.

Another explicitly very suitable polyvinylpyrrolidone (PVP) can be the polymer PVP K30, which is marketed by the company Ashland (ISP, POI Chemical). PVP K 30 is a polyvinylpyrrolidone that is very soluble in cold water and has the CAS number 9003-39-8. The molecular weight of PVP K 30 is about 40000 g/mol.

Other particularly well-suited polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115, which are available from BASF.

The use of film-forming hydrophilic polymers from the group of copolymers of polyvinylpyrrolidone has also led to particularly good and washfast color results.

In this context, vinylpyrrolidone-vinyl ester copolymers, such as those sold under the trademark Luviskol® (BASF), can be mentioned as particularly suitable film-forming, hydrophilic polymers. Luviskol® VA 64 and Luviskol® VA 73, each vinylpyrrolidone/vinyl acetate copolymers, are particularly preferred nonionic polymers.

Of the vinylpyrrolidone-comprising copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are very preferably used in the cosmetic compositions.

Vinylpyrrolidone-vinyl acetate copolymers are marketed by BASF SE under the name Luviskol® VA. For example, a VP/vinyl caprolactam/DMAPA acrylates copolymer is sold under the trade name Aquaflex® SF-40 by Ashland Inc. For example, a VP/DMAPA acrylates copolymer is marketed as Styleze CC-10 by Ashland and is a highly preferred vinylpyrrolidone-comprising copolymer.

Other suitable copolymers of polyvinylpyrrolidone may include those obtained by reacting N-vinylpyrrolidone with at least one further monomer selected from the group of V-vinylformamide, vinyl acetate, ethylene, propylene, acrylamide, vinylcaprolactam, vinylcaprolactone and/or vinyl alcohol.

Another suitable copolymer of vinylpyrrolidone is the polymer known under the INCI designation maltodextrin/VP copolymer.

Furthermore, intensively colored keratin material, especially hair, could be obtained with very good color fastness properties when a nonionic film-forming hydrophilic polymer was used as the film-forming hydrophilic polymer.

In a first embodiment, it may be preferred if the composition (B) comprises at least one nonionic, film-forming, hydrophilic polymer.

As contemplated herein, a non-ionic polymer is a polymer which, in a protic solvent—such as water, for example—does not carry structural units with permanent cationic or anionic groups under standard conditions, which must be compensated by counterions while maintaining electroneutrality. Cationic groups include, for example, quaternized ammonium groups but not protonated amines. Anionic groups include, for example, carboxylic and sulfonic acid groups.

Agents are particularly preferred which contain, as a nonionic, film-forming, hydrophilic polymer, at least one polymer selected from the group of
Polyvinylpyrrolidone,
copolymers of N-vinylpyrrolidone and vinyl esters of carboxylic acids comprising 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
Copolymers of N-vinylpyrrolidone with N,N-di(C1 to C4)alkylamino-(C2 to C4)alkyl acrylamide.

If copolymers of N-vinylpyrrolidone and vinyl acetate are used, it is again preferred if the molar ratio of the structural units included from the monomer N-vinylpyrrolidone to the structural units of the polymer included from the monomer vinyl acetate is in the range from 20 to 80 to 80 to 20, in particular from 30 to 70 to 60 to 40. Suitable copolymers of vinylpyrrolidone and vinyl acetate are available, for example, under the trademark Luviskol® VA 37, Luviskol® VA 55, Luviskol® VA 64 and Luviskol® VA 73 from BASF SE.

Another particularly preferred polymer is selected from polymers with the INCI designation VP/Methacrylamide/Vinyl Imidazole Copolymer, which are available, for example, under the trade name Luviset Clear from BASF SE.

Another particularly preferred nonionic, film-forming, hydrophilic polymer is a copolymer of N-vinylpyrrolidone and N,N-dimethylaminiopropylmethacrylamide, which is sold, for example, by ISP under the INCI designation VP/DMAPA Acrylates Copolymer, e.g. under the trade name Styleze® CC 10.

A cationic polymer as contemplated herein is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI name: Polyquatemium-69), which is marketed, for example, under the trade name AquaStyle® 300 (28-32% by weight active substance in ethanol-water mixture, molecular weight 350000) by the company ISP.

Other suitable film-forming hydrophilic polymers include
Vinylpyrrolidone-vinylimidazolium methochloride copolymers as offered under the names Luviquat® FC 370, FC 550 and the INCI name Polyquaternium-16 as well as FC 905 and HM 552,
Vinylpyrrolidone-vinylcaprolactam-acrylate terpolymers, such as those offered commercially with acrylic acid esters and acrylic acid amides as the third monomer building block, for example under the name Aquaflex® SF 40.

Polyquaternium-11 is the reaction product of diethyl sulfate with a copolymer of vinylpyrrolidone and dimethylaminoethyl methacrylate. Suitable commercial products are available, for example, under the names Dehyquart® CC 11 and Luviquat® PQ 11 PN from BASF SE or Gafquat 440, Gafquat 734, Gafquat 755 or Gafquat 755N from Ashland Inc.

Polyquaternium-46 is the reaction product of vinylcaprolactam and vinylpyrrolidone with methylvinylimidazolium methosulfate and is available, for example, under the name Luviquat® Hold from BASF SE. Polyquaternium-46 is preferably used in an amount of 1 to 5% by weight—based on the total weight of the cosmetic composition. It is particularly preferred that polyquaternium-46 is used in combination with a cationic guar compound. In fact, it is highly preferred that polyquaternium-46 be used in combination with a cationic guar compound and polyquaternium-11.

Suitable anionic film-forming hydrophilic polymers can be, for example, acrylic acid polymers, which can be in uncrosslinked or crosslinked form. Corresponding products are sold commercially under the trade names Carbopol 980, 981, 954, 2984 and 5984 by the company Lubrizol or under the names Synthalen M and Synthalen K by the company 3V Sigma (The Sun Chemicals, Inter Harz).

Examples of suitable film-forming hydrophilic polymers from the group of natural gums are xanthan gum, gellan gum, carob gum.

Suitable film-forming, hydrophilic polymers from the group of acrylamides are, for example, polymers prepared from monomers of (methy)acrylamido-C1-C4-alkyl-sulfonic acid or salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid, and/or poly-2-methylacrylamido-n-butanesulfonic acid.

Preferred polymers of poly(meth)arylamido-C₁-C₄-alkyl-sulfonic acids are crosslinked and at least 90% neutralized. These polymers can be crosslinked or uncrosslinked.

Cross-linked and fully or partially neutralized polymers of the poly-2-acrylamido-2-methylpropane sulfonic acid type are available under the INCI names “Ammonium Polyacrylamido-2-methyl-propanesulphonate” or “ammonium polyacryldimethyltauramide”.

Another preferred polymer of this type is the crosslinked poly-2-acrylamido-2methyl-propanesulfonic acid polymer sold by Clamant under the trade name Hostacerin AMPS, which is partially neutralized with ammonia.

In a further explicitly quite particularly preferred embodiment, a method as contemplated herein is described wherein the composition (A), (B) and/or the optionally applicable composition (C) comprises at least one anionic, film-forming, polymer.

In this context, the best results could be obtained when the composition (A), (B) and/or the optionally applicable composition (C) comprises at least one film-forming polymer comprising at least one structural unit of formula (P-I) and at least one structural unit of formula (P-II)

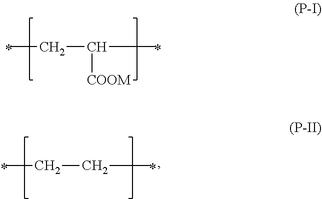

where
M represents a hydrogen atom or ammonium (NH4), sodium, potassium, ½ magnesium or ½ calcium.

When M represents a hydrogen atom, the structural unit of the formula (P-I) is based on an acrylic acid unit.

When M stands for an ammonium counterion, the structural unit of the formula (P-I) is based on the ammonium salt of acrylic acid.
When M represents a sodium counterion, the structural unit of the formula (P-I) is based on the sodium salt of acrylic acid.
When M stands for a potassium counterion, the structural unit of the formula (P-I) is based on the potassium salt of acrylic acid.
If M stands for a half equivalent of a magnesium counterion, the structural unit of the formula (P-I) is based on the magnesium salt of acrylic acid.
If M stands for a half equivalent of a calcium counterion, the structural unit of the formula (P-I) is based on the calcium salt of acrylic acid.

The film-forming polymer or polymers of the present disclosure are preferably used in specific ranges of amounts in the particular composition. In this context, it has proved particularly preferable for solving the problem as contemplated herein if the composition comprises—in each case based on its total weight—one or more film-forming polymers in a total amount of from 0.1 to 18.0% by weight, preferably from 1.0 to 16.0% by weight, more preferably from 5.0 to 14.5% by weight and very particularly preferably from 8.0 to 12.0% by weight.

### Application of the Compositions (A) and (B)

The method as contemplated herein comprises the application of both compositions (A) and (B) to the keratinous material. The two compositions (A) and (B) are two different compositions.

In one embodiment, it may be preferred to mix compositions (A) and (B) together prior to application to the keratin material so that the mixture of (A) and (B) is applied to the keratin material.

In a further embodiment, it may also be preferred to mix compositions (A) and (B) with a third previously described composition (C) prior to application to the keratin material, so that the mixture of (A) and (B) and (C) is applied to the keratin material.

In the context of a further particularly preferred embodiment, a device as contemplated herein is described relative to a method

wherein an application mixture is applied to the keratinous material, the application mixture comprising
prepared immediately before use by mixing the first composition (A) with the second composition (B), or the
prepared immediately before use by mixing the first composition (A) with the second composition (B) and the third composition (C).

Also possible and also as contemplated herein is the successive application of compositions (A) and (B), i.e. in this case composition (A) is first applied to the keratin material, allowed to act and, if necessary, rinsed out again. The composition (B) is then applied to the keratin material, allowed to act and, if necessary, rinsed out again.

In the context of this further embodiment, a method as contemplated herein is exemplified by the following steps:

(1) Application of the first composition (A) to the keratin material,
(2) Allowing the composition (A) to act on the keratin material for a period of 1 to 10 minutes, preferably 1 to 5 minutes,
(3) Rinsing the composition (A) out of the keratin material,
(4) Application of composition (B) to the keratin material,
(5) Allowing the composition (B) to act on the keratin material for a period of 1 to 10 minutes, preferably 1 to 5 minutes,
(6) Rinsing the composition (B) out of the keratin material.

As contemplated herein, the rinsing of the keratinous material with water in steps (3) and (6) of the method means that only water is used for the rinsing process, without the use of other compositions different from compositions (a) and (b).

In a step (1), the composition (A) is first applied to the keratin materials, especially human hair.

After application, the composition (A) is allowed to act on the keratin materials. In this context, exposure times of 10 seconds to 10 minutes, preferably 20 seconds to 5 minutes and most preferably 30 seconds to 2 minutes on the hair have proven to be particularly advantageous.

In a preferred embodiment of the method as contemplated herein, the composition (A) can now be rinsed from the keratin materials before the composition (B) is applied to the hair in the subsequent step.

In step (4), the composition (B) is now applied to the keratin materials. After application, the composition (B) is now left to act on the hair.

The method as contemplated herein allows the production of dyeings with particularly good intensity and color fastness even with short exposure times of the compositions (A) and (B). Exposure times of 10 seconds to 10 minutes, preferably 20 seconds to 5 minutes and most preferably 30 seconds to 3 minutes on the hair have proven to be particularly advantageous.

In step (6), the composition (B) is now rinsed out of the keratin material with water.

In another embodiment, a method as contemplated herein comprises the following steps in the order indicated:

(1) Application of the first composition (A) to the keratin material,
(2) Allowing the composition (A) to act on the keratin material for a period of 1 to 10 minutes, preferably 1 to 5 minutes,
(3) Rinsing the composition (A) out of the keratin material,
(4) Application of composition (B) to the keratin material,
(5) Allowing the composition (B) to act on the keratin material for a period of 1 to 10 minutes, preferably 1 to 5 minutes,
(6) Rinsing the composition (B) out of the keratin material.

Furthermore, if the optionally applicable third composition (C) is also applied to the keratin material, it can be applied in various ways.

One possibility is to mix composition (A) with composition (C) before application, and then apply the mixture of (A) and (C) to the keratin material.

Another option is to mix composition (B) with composition (C) before application, and then apply the mixture of (B) and (C) to the keratin material.

Furthermore, it is also encompassed by the present disclosure if all three compositions (A), (B) and (C) are mixed together prior to application and then this mixture of (A), (B) and (C) is applied to the keratin material.

In the context of a further embodiment, particularly preferred is a method as contemplated herein comprising the following steps:

(1) Application of the first composition (A) to the keratin material,
(2) Allowing the composition (A) to act on the keratin material for a period of 1 to 10 minutes, preferably 1 to 5 minutes,
(3) Rinsing the composition (A) out of the keratin material,
(4) Application of composition (B) to the keratin material,
(5) Allowing the composition (B) to act on the keratin material for a period of 1 to 10 minutes, preferably 1 to 5 minutes,
(6) Rinsing the composition (B) out of the keratin material.
(7) Application of composition (C) to the keratin material,
(8) Allowing the composition (C) to act on the keratin material for a period of 1 to 10 minutes, preferably 1 to 5 minutes,
(9) Rinsing the composition (C) out of the keratin material.

In the context of a further embodiment, particularly preferred is a method as contemplated herein comprising the following steps:

(1) Preparing an application mixture by mixing compositions (A) and (B)
(2) Apply the mixture of (A) and (B) to the keratin material,
(3) Allowing the mixture of (A) and (B) to act on the keratin material for a period of 1 to 10 minutes, preferably 1 to 5 minutes,
(4) Rinse the mixture out of the keratin material.

In the context of a further embodiment, particularly preferred is a method as contemplated herein comprising the following steps:

(1) Preparing an application mixture by mixing compositions (A) and (B)
(2) Apply the mixture of (A) and (B) to the keratin material,
(3) Allowing the mixture of (A) and (B) to act on the keratin material for a period of 1 to 10 minutes, preferably 1 to 5 minutes,
(4) Rinse the mixture out of the keratin material.
(5) Application of composition (C) to the keratin material
(6) Allowing the composition (C) to act on the keratin material for a period of 1 to 10 minutes, preferably 1 to 5 minutes, and
(7) Rinsing the composition (C) out of the keratin material.

In the context of a further embodiment, particularly preferred is a method as contemplated herein comprising the following steps:

(1) Preparing an application mixture by mixing compositions (A) and (B) and (C)
(2) Apply the mixture of (A) and (B) and (C) to the keratin material,
(3) Allowing the mixture of (A) and (B) and (C) to act on the keratin material for a period of 1 to 10 minutes, preferably 1 to 5 minutes,
(4) Rinse the mixture out of the keratin material.

### Multicomponent Packaging Unit (Kit-of-Parts)

To increase user convenience, all preparations required for the application method, in particular for the dyeing method, are provided to the user in the form of a multi-component packaging unit (kit-of-parts).

A second object of the present disclosure is a multi-component packaging unit (kit-of-parts) for treating keratinous material, comprising separately prepared
a first container comprising a first composition (A), and
a second container comprising a second composition (B), wherein the compositions (A) and (B) were disclosed in detail in the description of the first subject matter of the present disclosure,

Furthermore, the multi-component packaging unit as contemplated herein may also comprise a third packaging unit comprising a cosmetic preparation (C). The preparation (C) comprises, as described above, very particularly preferably at least one coloring compound.

In a very particularly preferred embodiment, the multi-component packaging unit (kit-of-parts) as contemplated herein comprises, separately assembled from one another—a third container comprising a third composition (C), the third composition (C) comprising at least one colorant compound selected from the group of pigments and/or direct dyes.

The colorant compounds from the group of pigments and direct dyes have already been disclosed in detail in the description of the first subject matter of the present disclosure.

With regard to the further preferred embodiments of the multicomponent packaging unit as contemplated herein, what has been said about the method as contemplated herein applies mutatis mutantis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A method for treating keratinous material in which the following are applied to the keratinous material:
a first composition (A) comprising
(A1) one or more organic C₁-C₆ alkoxy silanes and/or condensation products thereof, and
a second composition (B) comprising
(B1) a first cellulose and
(B2) a second cellulose different from the first cellulose (B1).

2. The method according to claim 1, wherein the first composition (A) comprises one or more organic C₁-C₆ alkoxy silanes (A1) of the formula (S-I) and/or (S-II),
R₁R₂N-L-Si(OR₃)ₐ(R₄)_{b} (S-I)
wherein
each of R₁, R₂ independently is a hydrogen atom or a C₁-C₆ alkyl group,
L is a linear or branched, divalent C₁-C₂₀ alkylene group,
R₃, R₄ are independent of each other and is a C₁-C₆ alkyl group,
a is an integer from 1 to 3,
b is an integer 3-a, and
(R₅O)_{c}(R₆)_{d}Si-(A′)ₑ-[NR₇-(A′)]_{f}-[O-(A″)]_{g}-[NR₈-(A′″)]_{h-Si}(R₆′)_{d}′(OR₅′)_{c}′ (S-II),
wherein
each of R₅, R₅′, R₅″, R₆, R₆′ and R₆″ independently is a C₁-C₆ alkyl group,
each of A, A′, A″, A′″ and A″″ independently is a linear or branched C₁-C₂₀ divalent alkylene group,
each of R₇ and R₈ independently is a hydrogen atom, a C₁-C₆ alkyl group, a hydroxy-C₁-C₆ alkyl group, a C₂-C₆ alkenylgroup, an amino-C₁-C₆ alkyl group or a group of formula (S-III),
(A″″)-Si(R₆″)_{d}″(OR₅″)_{c}″ (S-III),
wherein
c is an integer from 1 to 3,
d is an integer 3-c,
c′ is an integer from 1 to 3,
d′ is an integer 3-c,
c″ is an integer from 1 to 3,
d″ is an integer 3-c″,
e is 0 or 1,
f is 0 or 1,
g is 0 or 1,
h is 0 or 1,
with the proviso that at least one of the radicals and/or their condensation products from e, f, g and h is different from 0.

3. The method according to claim 1, wherein the first composition (A) comprises at least one C₁-C₆ organic alkoxysilane (A1) of formula (S-I) chosen from
(3-Aminopropyl)triethoxysilane;
(3-Aminopropyl)trimethoxysilane;
(2-Aminoethyl)triethoxysilane;
(2-Aminoethyl)trimethoxysilane;
(3-Dimethylaminopropyl)triethoxysilane;
(3-Dimethylaminopropyl)trimethoxysilane;
(2-dimethylaminoethyl)triethoxysilane;
(2-Dimethylaminoethyl)trimethoxysilane;
and/or their condensation products.

4. The method according to claim 1, wherein the first composition (A) comprises one or more organic C₁-C₆ alkoxy silanes (A1) of the formula (S-IV),
R₉Si(OR₁₀)ₖ(R₁₁)ₘ (S-IV),
where
R₉ is a C₁-C₁₂ alkyl group,
R₁₀ is a C₁-C₆ alkyl group,
R₁₁ is a C₁-C₆ alkyl group, and/or their condensation products,
k is an integer from 1 to 3, and
m is an integer 3-k.

5. The method according to claim 1, wherein the first composition (A) comprises at least one C₁-C₆ organic alkoxysilane (A1) of formula (S-IV) chosen from
Methyltrimethoxysilane;
Methyltriethoxysilane;
Ethyltrimethoxysilane;
Ethyltriethoxysilane;
Hexyltrimethoxysilane;
Hexyltriethoxysilane;
Octyltrimethoxysilane;
Octyltriethoxysilane;
Dodecyltrimethoxysilane;
Dodecyltriethoxysilane;
and/or their condensation products.

6. The method according to claim 1, wherein the composition (A) comprises—based on the total weight of the composition (A)—one or more organic C₁-C₆-alkoxysilanes (A1) and/or the condensation products thereof in a total amount of about 40.0 to about 99.0 wt.-%.

7. The method according to claim 1, wherein the first composition (A) comprises—based on the total weight of the composition (A)—about 0.01 to about 15.0% by weight of water.

8. The method according to claim 1, wherein the second composition (B) comprises
(B1) a first cellulose having at least one hydroxyethyl group, and
(B2) a second cellulose having at least one hydoxypropyl group.

9. The method according to claim 1, wherein the second composition (B) comprises
(B1) 2-hydroxyethyl cellulose; and
(B2) at least one cellulose chosen from 2-hydroxypropyl cellulose, 3-hydroxypropyl cellulose, 2-hydroxypropyl methyl cellulose and/or 3-hydroxypropyl methyl cellulose.

10. The method according to claim 1, wherein the second composition (B)—based on the total weight of the composition (B)—comprises (B1) about 0.1 to about 10.0% by weight of 2-hydroxyethyl cellulose.

11. The method according to claim 1, wherein the second composition (B) comprises, based on the total weight of the composition (B), (B2) one or more celluloses chosen from 2-hydroxypropyl cellulose, 3-hydroxypropyl cellulose, 2-hydroxypropyl methyl cellulose and/or 3-hydroxypropyl methyl cellulose in a total amount of about 0.1 to about 8.0% by weight.

12. The method according to claim 1, wherein the weight ratio of all celluloses (B1) in the composition (B) to all celluloses (B2) in the composition (B) is of from about 0.2 to about 5.0.

13. The method according to claim 1, wherein the second composition (B)—based on the total weight of the composition (B)—comprises about 30.0 to about 98.0% by weight of water.

14. The method according to claim 1, wherein the second composition (B) comprises at least one film-forming polymer comprising at least one structural unit of the formula (P-I) and at least one structural unit of the formula (P-II) 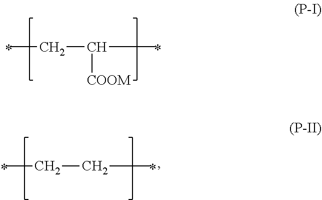
where
M is a hydrogen atom or ammonium (NH₄), sodium, potassium, ½ magnesium or ½ calcium.

15. The method according to claim 1, wherein a third composition (C) is applied on the keratinous material wherein the third composition comprises
at least one colorant compound chosen from pigments and/or direct dyes.

16. The method according to claim 15, wherein the second composition (B) and/or the third composition (C) comprises at least one inorganic pigment chosen from colored metal oxides, metal hydroxides, metal oxide hydrates, silicates, metal sulfides, complex metal cyanides, metal sulfates, bronze pigments and/or of colored mica- or mica-based pigments coated with at least one metal oxide and/or a metal oxychloride.

17. The method according to claim 15, wherein the second composition (B) and/or the third composition (C) comprises at least one colorant compound from the group of organic pigments chosen from carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

18. The method according to claim 1, wherein an application mixture is applied to the keratinous material and the application mixture is
prepared immediately before use by mixing the first composition (A) with the second composition (B), or
prepared immediately before use by mixing the first composition (A) with the second composition (B) and the third composition (C).

19. The method of claim 1, comprising the following steps:
(1) Preparing a first application mixture by mixing the first composition (A) with at least one further composition,
(2) Applying the first application mixture prepared in step (1) to the keratinous material,
(3) Allow-ing the first application mixture applied in step (2) to act,
(4) Washing the first application mixture and the keratinous material after exposure,
(5) Preparing a second application mixture by mixing the second composition (B) with a third composition (C),
(6) Applying the second application mixture prepared in step (5) to the keratinous material,
(7) Allowing the second application mixture applied in step (6) to act, and
(8) Washing the second application mixture and the keratinous material after exposure.

20. A multicomponent packaging unit (kit-of-parts) for treating keratinous material, comprising a separately prepared
first container comprising a first composition (A), and
second container comprising a second composition (B), wherein
said first composition (A) comprises (A1) one or more organic C₁-C₆ alkoxy silanes and/or condensation products thereof, and
said second composition (B) comprises (B1) a first cellulose and (B2) a second cellulose different from the first cellulose (B1), and optionally further comprising a separately assembled third container comprising a third composition (C), wherein said third composition (C) comprises at least one colorant compound chosen from pigments and/or direct dyes.

21. (canceled)
